(19) United States
(12) Patent Application Publication
(10) Pub. No.: US 2025/0152176 A1
(43) Pub. Date: May 15, 2025

(54) Devices and Methods for Occluding a Cerebral Aneurysm

(71) Applicant: Robert A. Connor, Wyoming, MN (US)

(72) Inventor: Robert A. Connor, Wyoming, MN (US)

(73) Assignee: Aneuclose LLC, Ham Lake, MN (US)

(21) Appl. No.: 19/023,514

(22) Filed: Jan. 16, 2025

Related U.S. Application Data
(63) Continuation-in-part of application No. 18/920,939, filed on Oct. 20, 2024.
(63) Continuation-in-part of application No. 18/760,322, filed on Jul. 1, 2024.
Application No. 18/920,939 is a continuation-in-part of application No. 18/760,322, filed on Jul. 1, 2024.
Application No. 18/760,322 is a continuation-in-part of application No. 18/674,996, filed on May 27, 2024, now abandoned.
Application No. 18/760,322 is a continuation-in-part of application No. 18/674,996, filed on May 27, 2024, now abandoned.
Application No. 18/674,996 is a continuation-in-part of application No. 18/613,053, filed on Mar. 21, 2024, now abandoned.
Application No. 18/613,053 is a continuation-in-part of application No. 17/970,510, filed on Oct. 20, 2022, now abandoned.
Application No. 18/674,996 is a continuation-in-part of application No. 18/613,053, filed on Mar. 21, 2024, now abandoned.
Application No. 18/613,053 is a continuation-in-part of application No. 18/519,055, filed on Nov. 26, 2023, now Pat. No. 12,004,750.
Application No. 18/613,053 is a continuation-in-part of application No. 18/519,055, filed on Nov. 26, 2023, now Pat. No. 12,004,750.
Application No. 18/519,055 is a continuation-in-part of application No. 18/135,153, filed on Apr. 15, 2023, now abandoned.
Application No. 18/519,055 is a continuation-in-part of application No. 18/374,602, filed on Sep. 28, 2023, now Pat. No. 11,944,316.
Application No. 18/374,602 is a continuation-in-part of application No. 18/135,153, filed on Apr. 15, 2023, now abandoned.
Application No. 18/135,153 is a continuation-in-part of application No. 17/970,510, filed on Oct. 20, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/374,602 is a continuation-in-part of application No. 18/135,153, filed on Apr. 15, 2023, now abandoned.
Application No. 18/135,153 is a continuation-in-part of application No. 17/970,510, filed on Oct. 20, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/135,153 is a continuation-in-part of application No. 17/970,510, filed on Oct. 20, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/829,313 is a continuation-in-part of application No. 17/485,390, filed on Sep. 25, 2021, now Pat. No. 11,471,164.
Application No. 17/485,390 is a continuation-in-part of application No. 17/476,845, filed on Sep. 16, 2021, now Pat. No. 11,583,289.
Application No. 17/476,845 is a continuation-in-part of application No. 17/472,674, filed on Sep. 12, 2021, now abandoned.
Application No. 17/472,674 is a continuation-in-part of application No. 17/467,680, filed on Sep. 7, 2021, now abandoned.
Application No. 17/467,680 is a continuation-in-part of application No. 17/466,497, filed on Sep. 3, 2021, now Pat. No. 11,357,511.
Application No. 17/466,497 is a continuation-in-part of application No. 17/353,652, filed on Jun. 21, 2021, now abandoned.
Application No. 17/353,652 is a continuation-in-part of application No. 17/220,002, filed on Apr. 1, 2021, now Pat. No. 11,464,518.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/211,446 is a continuation-in-part of application No. 16/693,267, filed on Nov. 23, 2019, now abandoned.
Application No. 16/693,267 is a continuation-in-part of application No. 16/660,929, filed on Oct. 23, 2019, now Pat. No. 11,471,163.
Application No. 16/693,267 is a continuation-in-part of application No. 16/660,929, filed on Oct. 23, 2019, now Pat. No. 11,471,163.
Application No. 16/660,929 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 15/865,822, filed on Jan. 9, 2018, now Pat. No. 10,716,573.
Application No. 15/865,822 is a continuation-in-part of application No. 15/081,909, filed on Mar. 27, 2016, now abandoned.
Application No. 15/081,909 is a continuation-in-part of application No. 14/526,600, filed on Oct. 29, 2014, now abandoned.
Application No. 14/526,600 is a continuation-in-part of application No. 15/080,915, filed on Mar. 25, 2016, now Pat. No. 10,028,747.
Application No. 15/080,915 is a continuation-in-part of application No. 14/526,600, filed on Oct. 29, 2014, now abandoned.
Application No. 15/081,909 is a continuation-in-part of application No. 14/526,600, filed on Oct. 29, 2014, now abandoned.
Application No. 15/080,915 is a continuation-in-part of application No. 14/526,600, filed on Oct. 29, 2014, now abandoned.
Application No. 14/526,600 is a continuation-in-part of application No. 12/989,048, filed on Oct. 21, 2010, now Pat. No. 8,974,487.
(60) Provisional application No. 63/119,774, filed on Dec. 1, 2020.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/720,173, filed on Aug. 21, 2018.
(60) Provisional application No. 62/589,754, filed on Nov. 22, 2017.
(60) Provisional application No. 62/472,519, filed on Mar. 16, 2017.
(60) Provisional application No. 62/589,754, filed on Nov. 22, 2017.
(60) Provisional application No. 62/472,519, filed on Mar. 16, 2017.
(60) Provisional application No. 62/444,860, filed on Jan. 11, 2017.
(60) Provisional application No. 61/897,245, filed on Oct. 30, 2013.
(60) Provisional application No. 61/126,047, filed on May 1, 2008.
(60) Provisional application No. 61/126,027, filed on May 1, 2008.

(51) Int. Cl. A61B 17/12; A61B 17/00

(52) U.S. Cl. CPC A61B 17/12113; A61B 17/00234; A61B 17/1214; A61B 17/12168; A61B 2017/00238; A61B 2017/00292; A61B 2017/00862; A61B 2017/00951; A61B 2017/1205

(57) ABSTRACT

This invention is a device to occlude a cerebral aneurysm which includes: a longitudinal lumen that is inserted into the parent blood vessel of an aneurysm; a flexible expandable member (such as a net or mesh) which is expanded within the aneurysm sack by the insertion of embolic members into that flexible expandable member; and a resilient expandable member (such as a cylindrical stent or ring stent) that is attached to a central portion of the flexible expandable member and expanded within the aneurysm sack in order to keep the flexible expandable member from slipping out of the aneurysm sack.

### CROSS-REFERENCE TO RELATED APPLICATIONS

This application is a continuation-in-part of U.S. patent application Ser. No. 18/920,939 filed on 2024 Oct. 20 and a continuation-in-part of U.S. patent application Ser. No. 18/760,322 filed on 2024 Jul. 1. U.S. patent application Ser. No. 18/920,939 was a continuation-in-part of U.S. patent application Ser. No. 18/760,322 filed on 2024 Jul. 1 and a continuation-in-part of U.S. patent application Ser. No. 18/674,996 filed on 2024 May 27. U.S. patent application Ser. No. 18/760,322 was a continuation-in-part of U.S. patent application Ser. No. 18/674,996 filed on 2024 May 27, a continuation-in-part of U.S. patent application Ser. No. 18/613,053 filed on 2024 Mar. 21, and a continuation-in-part of U.S. patent application Ser. No. 17/970,510 filed on 2022 Oct. 20.

U.S. patent application Ser. No. 18/674,996 was a continuation-in-part of U.S. Pat. No. 18,613,053 filed on 2024 Mar. 21 and a continuation-in-part of U.S. patent application Ser. No. 18/519,055 filed on 2023 Nov. 26. U.S. patent application Ser. No. 18/613,053 was a continuation-in-part of U.S. patent application Ser. No. 18/519,055 filed on 2023 Nov. 26 and a continuation-in-part of U.S. patent application Ser. No. 18/135,153 filed on 2023 Apr. 15.

U.S. patent application Ser. No. 18/519,055 was a continuation-in-part of U.S. patent application Ser. No. 18/374,602 filed on 2023 Sep. 28, a continuation-in-part of U.S. patent application Ser. No. 18/135,153 filed on 2023 Apr. 15, a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31.

U.S. patent application Ser. No. 18/374,602 was a continuation-in-part of U.S. patent application Ser. No. 18/135,153 filed on 2023 Apr. 15, a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31.

U.S. patent application Ser. No. 18/135,153 was a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31. U.S. patent application Ser. No. 17/970,510 was a continuation-in-part of U.S. patent application Ser. No. 17/965,502 filed on 2022 Oct. 13, a continuation-in-part of U.S. patent application Ser. No. 17/829,313 filed on 2022 May 31, and a continuation-in-part of U.S. patent application Ser. No. 17/476,845 filed on 2021 Sep. 16.

U.S. patent application Ser. No. 17/829,313 was a continuation-in-part of U.S. patent application Ser. No. 17/485,390 filed on 2021 Sep. 25, was a continuation-in-part of U.S. patent application Ser. No. 17/476,845 filed on 2021 Sep. 16, was a continuation-in-part of U.S. patent application Ser. No. 17/472,674 filed on 2021 Sep. 12, was a continuation-in-part of U.S. patent application Ser. No. 17/467,680 filed on 2021 Sep. 7, was a continuation-in-part of U.S. patent application Ser. No. 17/466,497 filed on 2021 Sep. 3, was a continuation-in-part of U.S. patent application Ser. No. 17/353,652 filed on 2021 Jun. 21, was a continuation-in-part of U.S. patent application Ser. No. 17/220,002 filed on 2021 Apr. 1, was a continuation-in-part of U.S. patent application Ser. No. 17/214,827 filed on 2021 Mar. 27, was a continuation-in-part of U.S. patent application Ser. No. 17/211,446 filed on 2021 Mar. 24, was a continuation-in-part of U.S. patent application Ser. No. 16/693,267 filed on 2019 Nov. 23, and was a continuation-in-part of U.S. patent application Ser. No. 16/660,929 filed on 2019 Oct. 23.

U.S. patent application Ser. No. 17/220,002 was a continuation-in-part of U.S. patent application Ser. No. 17/214,827 filed on 2021 Mar. 27. U.S. patent application Ser. No. 17/220,002 was a continuation-in-part of U.S. patent application Ser. No. 17/211,446 filed on 2021 Mar. 24. U.S. patent application Ser. No. 17/220,002 claimed the priority benefit of U.S. provisional patent application 63/119,774 filed on 2020 Dec. 1. U.S. patent application Ser. No. 17/220,002 was a continuation-in-part of U.S. patent application Ser. No. 16/693,267 filed on 2019 Nov. 23. U.S. patent application Ser. No. 17/220,002 was a continuation-in-part of U.S. patent application Ser. No. 16/660,929 filed on 2019 Oct. 23.

U.S. patent application Ser. No. 16/693,267 was a continuation-in-part of U.S. patent application Ser. No. 16/660,929 filed on 2019 Oct. 23. U.S. patent application Ser. No. 16/693,267 claimed the priority benefit of U.S. provisional patent application 62/794,609 filed on 2019 Jan. 19. U.S. patent application Ser. No. 16/693,267 claimed the priority benefit of U.S. provisional patent application 62/794,607 filed on 2019 Jan. 19. U.S. patent application Ser. No. 16/693,267 was a continuation-in-part of U.S. patent application Ser. No. 16/541,241 filed on 2019 Aug. 15. U.S. patent application Ser. No. 16/693,267 was a continuation-in-part of U.S. patent application Ser. No. 15/865,822 filed on 2018 Jan. 9 which issued as U.S. Pat. No. 10,716,573 on 2020 Jul. 21. U.S. patent application Ser. No. 16/693,267 was a continuation-in-part of U.S. patent application Ser. No. 15/861,482 filed on 2018 Jan. 3.

U.S. patent application Ser. No. 16/660,929 claimed the priority benefit of U.S. provisional patent application 62/794,609 filed on 2019 Jan. 19. U.S. patent application Ser. No. 16/660,929 claimed the priority benefit of U.S. provisional patent application 62/794,607 filed on 2019 Jan. 19. U.S. patent application Ser. No. 16/660,929 was a continuation-in-part of U.S. patent application Ser. No. 16/541,241 filed on 2019 Aug. 15. U.S. patent application Ser. No. 16/660,929 was a continuation-in-part of U.S. patent application Ser. No. 15/865,822 filed on 2018 Jan. 9 which issued as U.S. Pat. No. 10,716,573 on 2020 Jul. 21. U.S. patent application Ser. No. 16/660,929 was a continuation-in-part of U.S. patent application Ser. No. 15/861,482 filed on 2018 Jan. 3.

U.S. patent application Ser. No. 16/541,241 claimed the priority benefit of U.S. provisional patent application 62/794,609 filed on 2019 Jan. 19. U.S. patent application Ser. No. 16/541,241 claimed the priority benefit of U.S. provisional patent application 62/794,607 filed on 2019 Jan. 19. U.S. patent application Ser. No. 16/541,241 claimed the priority benefit of U.S. provisional patent application 62/720,173 filed on 2018 Aug. 21. U.S. patent application Ser. No. 16/541,241 was a continuation-in-part of U.S. patent application Ser. No. 15/865,822 filed on 2018 Jan. 9 which issued as U.S. Pat. No. 10,716,573 on 2020 Jul. 21

U.S. patent application Ser. No. 15/865,822 claimed the priority benefit of U.S. provisional patent application 62/589,754 filed on 2017 Nov. 22. U.S. patent application Ser. No. 15/865,822 claimed the priority benefit of U.S. provisional patent application 62/472,519 filed on 2017 Mar. 16. U.S. patent application Ser. No. 15/865,822 was a continuation-in-part of U.S. patent application Ser. No. 15/081,909 filed on 2016 Mar. 27. U.S. patent application Ser. No. 15/865,822 was a continuation-in-part of U.S. patent application Ser. No. 14/526,600 filed on 2014 Oct. 29.

U.S. patent application Ser. No. 15/861,482 claimed the priority benefit of U.S. provisional patent application 62/589,754 filed on 2017 Nov. 22. U.S. patent application Ser. No. 15/861,482 claimed the priority benefit of U.S. provisional patent application 62/472,519 filed on 2017 Mar. 16. U.S. patent application Ser. No. 15/861,482 claimed the priority benefit of U.S. provisional patent application 62/444,860 filed on 2017 Jan. 11. U.S. patent application Ser. No. 15/861,482 was a continuation-in-part of U.S. patent application Ser. No. 15/080,915 filed on 2016 Mar. 25 which issued as U.S. Pat. No. 10,028,747 on 2018 Jul. 24. U.S. patent application Ser. No. 15/861,482 was a continuation-in-part of U.S. patent application Ser. No. 14/526,600 filed on 2014 Oct. 29.

U.S. patent application Ser. No. 15/081,909 was a continuation-in-part of U.S. patent application Ser. No. 14/526,600 filed on 2014 Oct. 29. U.S. patent application Ser. No. 15/080,915 was a continuation-in-part of U.S. patent application Ser. No. 14/526,600 filed on 2014 Oct. 29. U.S. patent application Ser. No. 14/526,600 claimed the priority benefit of U.S. provisional patent application 61/897,245 filed on 2013 Oct. 30. U.S. patent application Ser. No. 14/526,600 was a continuation-in-part of U.S. patent application Ser. No. 12/989,048 filed on 2010 Oct. 21 which issued as U.S. Pat. No. 8,974,487 on 2015 Mar. 10. U.S. patent application Ser. No. 12/989,048 claimed the priority benefit of U.S. provisional patent application 61/126,047 filed on 2008 May 1. U.S. patent application Ser. No. 12/989,048 claimed the priority benefit of U.S. provisional patent application 61/126,027 filed on 2008 May 1.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to aneurysm occlusion devices and methods.

### INTRODUCTION

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Even among those who survive, approximately one-half suffer significant and permanent deterioration of brain function. Better alternatives for cerebral aneurysm treatment are needed.

### REVIEW OF THE RELEVANT ART

U.S. patent application No. 20010000797 (Mazzocch, May 3, 2001, “Method and Apparatus for Occluding Aneurysms”) discloses aneurysm occlusion devices formed from a resilient metal fabric. U.S. patent application No. 20010034531 (Ho et al., Oct. 25, 2001, “Bioactive Three Loop Coil”) discloses an implantable medical device for obstructing a neck portion of a vascular aneurysm. U.S. patent application No. 20020026210 (Abdel-Gawwad, Feb. 28, 2002, “Endovascular Aneurysm Treatment Device and Method”) discloses a self-expanding frame which is mounted on the distal end of a shaft. U.S. patent application No. 20020165572 (Saadat, Nov. 7, 2002, “Embolic Balloon”) discloses an embolic balloon assembly. U.S. patent application No. 20030028209 (Teoh et al., Feb. 6, 2003, “Expandable Body Cavity Liner Device”) and U.S. patent application No. 20040098027 (Teoh et al., May 20, 2004, “Expandable Body Cavity Liner Device”) disclose an aneurysm treatment device with a liner.

U.S. patent application No. 20030040772 (Hyodoh et al., Feb. 27, 2003, “Delivery Devices”) discloses self-expandable, woven intravascular devices for use as stents (both straight and tapered), filters (both temporary and permanent) and occluders for insertion and implantation into a variety of anatomical structures. U.S. patent application No. 20030139802 (Wulfman et al., Jul. 24, 2003, “Medical Device”) discloses a radially expandable device for use in the occlusion and repair of an undesired dilation in a vessel. U.S. patent application No. 20030171739 (Murphy et al., Sep. 11, 2003, “Detachable Aneurysm Neck Bridge”) discloses a neck bridge for bridging the neck of an aneurysm includes a junction region, a number of radially extending array elements attached to the junction region, and a cover attached to one or both of the junction region and an array element.

U.S. patent application No. 20030187473 (Berenstein et al., Oct. 2, 2003, “Expandable Body Cavity Liner Device”) discloses aneurysm treatment device for treating aneurysms of various shapes and sizes. U.S. patent application No. 20030195553 (Wallace et al., Oct. 16, 2003, “System and Method for Retaining Vaso-Occlusive Devices Within an Aneurysm”) discloses a device with a mesh-like structure that is integrally composed of a shape-memory alloy such as NiTi. U.S. patent application 20040254625 (Stephens et al., Dec. 16, 2004, “Inflatable Implant”) discloses an inflatable implant suitable for placement in the human body. U.S. patent application No. 20050043786 (Chu et al., Feb. 24, 2005, “Methods and Apparatus for Treatment of Aneurysmal Tissue”) discloses methods and apparatus for aiding aneurysm repair, including contracting if the aneurysmal site shrinks or contracts.

U.S. patent application No. 20050277978 (Greenhalgh, Dec. 15, 2005, “Three-Dimensional Coils for Treatment of Vascular Aneurysms”) discloses a coil formed from filamentary members interlaced to form a three-dimensional substrate. U.S. patent application No. 20060116709 (Sepetka et al., Jun. 1, 2006, “Aneurysm Treatment Devices and Methods”) discloses an aneurysm treatment device for in situ treatment of aneurysms comprising a collapsible member having a first shape wherein the first shape is an expanded geometric configuration, and a second shape, wherein the second shape is a collapsed configuration that is loadable into a catheter. U.S. patent application No. 20060116713 (Sepetka et al., Jun. 1, 2006, “Aneurysm Treatment Devices and Methods”) discloses an aneurysm treatment device for in situ treatment of aneurysms comprising an occlusion device having a flexible, longitudinally extending elastomeric matrix member that assumes a non-linear shape to conformally fill a targeted vascular site.

U.S. patent application No. 20060155323 (Porter et al., Jul. 13, 2006, “Intra-Aneurysm Devices”) and U.S. Pat. No. 10,265,075 (Porter et al., Apr. 23, 2019, “Intra-Aneurysm Devices”) disclose a device with an upper member that sits against a dome of an aneurysm, a lower member that sits in the neck of the aneurysm, and a means of adjusting the overall dimensions of the device. U.S. patent application No. 20070100426 (Rudakov et al., May 3, 2007, “Medical Device”) discloses inserting a medical device such that it is at least partially located in the first artery and is at least partially located in the third artery, and then expanding the medical device. U.S. patent application 20070150045 (Ferrera, Jun. 28, 2007, “Methods and Apparatus for Treating Aneurysms and Other Vascular Defects”) discloses devices and methods for placing a barrier across the neck of a vascular aneurysm.

U.S. patent application No. 20070162106 (Evans et al., Jul. 12, 2007, “System and Methods for Endovascular Aneurysm Treatment”) discloses methods and systems for treating aneurysms using filling structures filled with a curable medium. U.S. patent application No. 20070219578 (Solar et al., Sep. 20, 2007, “Aneurysm Coil Delivery System”) discloses devices and methods for treating an aneurysm with a single unit having an access element and an occlusion element, the access element providing access to the aneurysm for introducing treatment objects such as coils therethrough while the occlusion element blocks the treatment objects from protruding into the vessel. U.S. patent application No. 20070265656 (Amplatz et al., Nov. 15, 2007, “Multi-Layer Braided Structures for Occluding Vascular Defects”) discloses a collapsible medical device and associated methods of occluding an abnormal opening in, for example, a body organ, wherein the medical device is shaped from plural layers of a heat-treatable metal fabric.

U.S. patent application No. 20070270903 (Davis III et al., Nov. 22, 2007, “Stretch Resistant Embolic Coil Delivery System with Combined Mechanical and Pressure Release Mechanism”) discloses a medical device for placing an embolic device at a predetermined site within a vessel of the body including a delivery catheter and a flexible pusher member slidably disposed within the lumen of the catheter. U.S. patent application No. 20080281350 (Sepetka et al., Nov. 13, 2008, “Aneurysm Occlusion Devices”) discloses an implantable occlusion device for bridging the neck of an aneurysm comprising a biocompatible matrix. U.S. patent application No. 20090112249 (Miles et al., Apr. 30, 2009, “Medical Device for Modification of Left Atrial Appendage and Related Systems and Methods”) discloses devices, systems and methods for modifying an atrial appendage with a body that is collapsible and self-expanding.

U.S. patent application No. 20090227976 (Calabria et al., Sep. 10, 2009, “Multiple Biocompatible Polymeric Strand Aneurysm Embolization System and Method”) discloses a catheter to transport a biocompatible polymeric strand to an aneurysm. U.S. patent application No. 20090287294 (Rosqueta et al., Nov. 19, 2009, “Braid-Ball Embolic Devices”) discloses embolic implants, delivery systems, and methods of manufacture and delivery. U.S. patent application No. 20090318948 (Linder et al., Dec. 24, 2009, “Device, System and Method for Aneurysm Embolization”) discloses an apparatus, method, and system for treatment of aneurysms including a handle, a controller coupled to the handle and a catheter coupled to the handle. U.S. Pat. No. 8,092,819 (Ruane et al., Jan. 10, 2012, “Implantable Medical Device Coated with a Bioactive Agent”) discloses an implantable medical device comprising a bioactive agent and poly(alkyl cyanoacrylate) polymer. U.S. Pat. No. 8,142,456 (Rosqueta et al., Mar. 27, 2012, “Braid-Ball Embolic Devices”) discloses implant designs with low profile compressibility for delivery to neurovasculature, while maintaining other necessary features such as density for occlusion purposes and desirable radial strength characteristics.

U.S. patent application No. 20120239074 (Aboytes et al., Sep. 20, 2012, “Devices and Methods for the Treatment of Vascular Defects”), U.S. patent application No. 20130116722 (Aboytes et al., May 9, 2013, “Devices and Methods for the Treatment of Vascular Defects”), U.S. Pat. No. 8,998,947 (Aboytes et al., Apr. 7, 2015, “Devices and Methods for the Treatment of Vascular Defects”), U.S. patent application No. 20150209050 (Aboytes et al., Jul. 30, 2015, “Devices and Methods for the Treatment of Vascular Defects”), U.S. patent application No. 20150272590 (Aboytes et al., Oct. 1, 2015, “Devices and Methods for the Treatment of Vascular Defects”), U.S. patent application 20150342613 (Aboytes et al., Dec. 3, 2015, “Devices and Methods for the Treatment of Vascular Defects”), U.S. patent application No. 20160262766 (Aboytes et al., Sep. 15, 2016, “Devices and Methods for the Treatment of Vascular Defects”), U.S. patent application No. 20180125501 (Aboytes et al., May 10, 2018, “Devices and Methods for the Treatment of Vascular Defects”), U.S. patent application 20180132859 (Aboytes et al., May 17, 2018, “Devices and Methods for the Treatment of Vascular Defects”), U.S. patent application No. 20180132862 (Aboytes et al., May 17, 2018, “Devices and Methods for the Treatment of Vascular Defects”), and U.S. Pat. No. 10,064,627 (Aboytes et al., Sep. 4, 2018, “Devices and Methods for the Treatment of Vascular Defects”) disclose an apparatus with an insertion portion and an expandable implant, wherein the implant is movable between a first configuration in which the first portion and the second portion are substantially linearly aligned and a second configuration in which the second portion at least partially overlaps the first portion.

U.S. patent application No. 20130245667 (Marchand et al., Sep. 19, 2013, “Filamentary Devices and Treatment of Vascular Defects”) discloses devices and methods for treatment of a patient's vasculature with some embodiments configured for delivery with a microcatheter. U.S. patent application No. 20140052233 (Cox et al., Feb. 20, 2014, “Methods and Devices for Treatment of Vascular Defects”) discloses providing an expandable wire body support structure having a low profile radially constrained state, an expanded relaxed state with a substantially spherical or globular configuration having a smooth outer surface, and a porous permeable layer. U.S. Pat. No. 8,690,907 (Janardhan et al., Apr. 8, 2014, “Vascular Treatment Methods”) discloses vascular treatment and methods including a plurality of self-expanding bulbs and a hypotube including interspersed patterns of longitudinally spaced rows of kerfs.

U.S. Pat. No. 8,715,338 (Frid, May 6, 2014, “Luminal Endoprosthesis for the Occlusion of an Aneurysm and Method of Manufacturing Such an Endoprosthesis”) discloses a tubular armature that can expand radially from a compressed state to an expanded state. U.S. patent application 20140135812 (Divino et al., May 15, 2014, “Occlusive Devices”) and U.S. Pat. No. 10,327,781 (Divino et al., Jun. 25, 2019, “Occlusive Devices”) disclose a device with at least one expandable structure adapted to transition from a compressed configuration to an expanded configuration when released into the aneurysm. U.S. Pat. No. 8,771,294 (Sepetka et al., Jul. 8, 2014, “Aneurysm Treatment Devices and Methods”) discloses an aneurysm treatment device for in situ treatment of aneurysms comprising a collapsible member having a first shape wherein the first shape is an expanded geometric configuration, and a second shape, wherein the second shape is a collapsed configuration that is loadable into a catheter.

U.S. patent application No. 20140330299 (Rosenbluth et al., Nov. 6, 2014, “Embolic Occlusion Device and Method”) discloses an occlusion device including a tubular braided member having a first end and a second end and extending along a longitudinal axis, the tubular braided member having a repeating pattern of larger diameter portions and smaller diameter portions arrayed along the longitudinal axis, and at least one metallic coil member extending coaxially along at least a portion of the braided member. U.S. Pat. No. 9,078,658 (Hewitt et al., Jul. 14, 2015, “Filamentary Devices for Treatment of Vascular Defects”) discloses a self-expanding resilient permeable shell having a radially constrained elongated state configured for delivery within a catheter lumen, an expanded state with a globular and longitudinally shortened configuration relative to the radially constrained state, and a plurality of elongate filaments which are woven together.

U.S. patent application No. 20150313605 (Griffin, Nov. 5, 2015, “Occlusion Device”) and U.S. Pat. No. 10,130,372 (Griffin, Nov. 20, 2018, “Occlusion Device”) disclose an occlusion device for intrasaccular implantation and/or vascular occlusion comprising: (a) a substantially solid marker having a proximal end, and a distal end; and (b) a low profile resilient mesh body attached to the distal end of the marker. U.S. Pat. No. 9,198,668 (Theobald et al., Dec. 1, 2015, “Cerebral Aneurysm Closure Device”) discloses a closure device for blocking blood flow into an aneurysm. U.S. patent application No. 20160045201 (Rosenbluth et al., Feb. 18, 2016, “Blood Flow Disruption Devices and Methods for the Treatment of Vascular Defects”) discloses a blood flow disruption device for embolizing blood flowing into a vascular defect between a proximal vascular segment and a distal vascular segment.

U.S. Pat. No. 9,393,022 (Becking et al., Jul. 19, 2016, “Two-Stage Deployment Aneurysm Embolization Devices”) discloses implants which are deployed in two stages. U.S. patent application No. 20160249934 (Hewitt et al., Sep. 1, 2016, “Filamentary Devices for Treatment of Vascular Defects”) discloses implants made of woven braided mesh having a variable mesh density. U.S. Patent application Ser. No. 20160249935 (Hewitt et al., Sep. 1, 2016, “Devices for Therapeutic Vascular Procedures”), U.S. patent application No. 20160367260 (Hewitt et al., Dec. 22, 2016, “Devices for Therapeutic Vascular Procedures”), U.S. Pat. No. 9,629,635 (Hewitt et al., Apr. 25, 2017, “Devices for Therapeutic Vascular Procedures”), and U.S. patent application No. 20170128077 (Hewitt et al., May 11, 2017, “Devices for Therapeutic Vascular Procedures”) disclose methods and devices for removing a thrombus.

U.S. patent application No. 20160249937 (Marchand et al., Sep. 1, 2016, “Multiple Layer Filamentary Devices for Treatment of Vascular Defects”) and U.S. Pat. No. 9,918,720 (Marchand et al., Mar. 20, 2018, “Multiple Layer Filamentary Devices for Treatment of Vascular Defects”) disclose a permeable shell and inner structure configured to occlude blood flow therethrough. U.S. Pat. No. 9,492,174 (Hewitt et al., Nov. 15, 2016, “Filamentary Devices for Treatment of Vascular Defects”) discloses a self-expanding permeable shell having a radially constrained elongated state configured for delivery within a catheter lumen, an expanded state with a globular and longitudinally shortened configuration relative to the radially constrained state, and a plurality of elongate filaments that are woven together. U.S. Pat. No. 9,592,363 (Griffin et al., Mar. 14, 2017, “Medical Device”) discloses a medical device with a shaft having an elongated inner member and an elongated tubular reinforcing member disposed over at least a portion of the inner member.

U.S. Pat. No. 9,597,087 (Marchand et al., Mar. 21, 2017, “Filamentary Devices for Treatment of Vascular Defects”) discloses a permeable shell configured to occlude blood flow. U.S. patent application No. 20170095254 (Hewitt et al., Apr. 6, 2017, “Filamentary Devices for Treatment of Vascular Defects”) discloses a self-expanding permeable shell having a radially constrained elongated state configured for delivery within a catheter lumen, an expanded state with a globular and longitudinally shortened configuration relative to the radially constrained state, and a plurality of elongate filaments that are woven together. U.S. patent application No. 20170156734 (Griffin, Jun. 8, 2017, “Occlusion Device”) discloses an occlusion device for implantation into a body lumen or aneurysm comprising, a continuous compressible mesh structure comprising axial mesh carriages configured end to end, wherein each end of each carriage is a pinch point in the continuous mesh structure.

U.S. Pat. No. 9,687,245 (Molaei et al., Jun. 27, 2017, “Occlusive Devices and Methods of Use”) discloses a catheter with a proximal end, a distal end, and a lumen extending between the proximal and distal ends, wherein the proximal end can have a self-expanding distal section, and the distal section can have a coil portion. U.S. Pat. No. 9,713,475 (Divino et al., Jul. 25, 2017, “Embolic Medical Devices”) discloses an occlusive device with an elongate member having opposing first and second side edges extending longitudinally along the member and a member width. U.S. patent application 20170245862 (Cox et al., Aug. 31, 2017, “Methods and Devices for Treatment of Vascular Defects”) discloses a device with a first hub, a second hub, a support structure including a plurality of struts disposed between the first hub and the second hub, and a layer of material disposed over the plurality of struts.

U.S. patent application No. 20170258473 (Plaza et al., Sep. 14, 2017, “Systems and Methods for Delivery of Stents and Stent-Like Devices”) discloses an elongate tubular member having a lumen, an expandable stent, and a delivery device. U.S. Pat. No. 9,763,815 (Strauss et al., Sep. 19, 2017, “Protuberant Aneurysm Bridging Device Deployment Method”) discloses placing an aneurysm bridging device in the neurovasculature of a patient by advancing the aneurysm bridging device in a small-diameter configuration a delivery catheter to a target region within the neurovasculature and securing the distal region of the aneurysm bridging device to the neurovasculature. U.S. Pat. No. 9,801,744 (Berez et al., Oct. 31, 2017, “Methods and Apparatus for Luminal Stenting”) discloses flexible implantable occluding devices that can navigate the tortuous vessels of the neurovasculature.

U.S. patent application No. 20180000489 (Marchand et al., Jan. 4, 2018, “Filamentary Devices for Treatment of Vascular Defects”) discloses a self-expanding resilient permeable shell having a plurality of elongate resilient filaments with a woven structure. U.S. patent application 20180036012 (Aboytes et al., Feb. 8, 2018, “Devices, Systems, and Methods for the Treatment of Vascular Defects”) discloses an occlusion device that includes a proximal portion, a distal portion, and an intermediate portion extending between the proximal portion and the distal portion. U.S. Pat. No. 9,901,435 (Janardhan et al., Feb. 27, 2018, “Longitudinally Variable Vascular Treatment Devices”) discloses a woven structure including a plurality of bulbs that may be self-expanding, a hypotube, for example including interspersed patterns of longitudinally spaced rows of kerfs.

U.S. Pat. No. 9,955,976 (Hewitt et al., May 1, 2018, “Filamentary Devices for Treatment of Vascular Defects”) discloses implants made of woven braided mesh having a variable mesh density. U.S. Pat. No. 9,980,733 (Badruddin et al., May 29, 2018, “System for and Method of Treating Aneurysms”) discloses an apparatus for treating an aneurysm in a blood vessel via a wire to be advanced within a tube and an occlusion element disposed on the wire. U.S. patent application 20180206849 (Hewitt et al., Jul. 26, 2018, “Filamentary Devices for the Treatment of Vascular Defects”) discloses a self-expanding resilient permeable shell having a radially constrained state and an expanded state with a globular, axially shortened configuration. U.S. Pat. No. 10,034,966 (Bettinger et al., Jul. 31, 2018, “Coated Vaso-Occlusive Device and Methods for Treatment of Aneurysms”) discloses a method for the treatment of intracranial aneurysms comprising inserting an embolism coil coated with a polymeric coating.

U.S. Pat. No. 10,076,429 (Sahl, Sep. 18, 2018, “Membrane Implant for Treatment of Cerebral Artery Aneurysms”) discloses a membrane implant for treatment of cerebral artery aneurysms. U.S. Patent application 20180271540 (Merritt et al., Sep. 27, 2018, “Systems and Methods for Embolization of Body Structures”) discloses a self-expanding permeable shell having a proximal end, a distal end, and a longitudinal axis, the shell comprising a plurality of elongate resilient filaments having a braided structure. U.S. patent application No. 20180303486 (Rosenbluth et al., Oct. 25, 2018, “Embolic Occlusion Device and Method”) discloses a tubular braided member having a first end and a second end and extending along a longitudinal axis, the tubular braided member having a repeating pattern of larger diameter portions and smaller diameter portions arrayed along the longitudinal axis, and at least one metallic coil member extending coaxially along at least a portion of the braided member.

U.S. patent application No. 20190046209 (Plaza et al., Feb. 14, 2019, “Delivery and Detachment Systems and Methods for Vascular Implants”) discloses a system for delivering an implant device to a vascular site in a patient via a delivery pusher apparatus. U.S. patent application No. 20190053810 (Griffin, Feb. 21, 2019, “Occlusion Device”) discloses an occlusion device comprising: (a) a substantially solid marker band having an inner and outer diameter, a proximal end, and a distal end; and (b) a resilient mesh body attached within the marker. U.S. patent application No. 20190059909 (Griffin, Feb. 28, 2019, “Occlusion Device”) discloses an occlusion device for intrasaccular implantation and/or vascular occlusion comprising: (a) a substantially solid marker having a proximal end, and a distal end; and (b) a low profile resilient mesh body attached to the distal end of the marker.

U.S. Pat. No. 10,314,593 (Bardsley et al., Jun. 11, 2019, “Occlusive Devices”) discloses an implant with single or dual layer braided body having a variable porosity. U.S. Pat. No. 10,383,749 (Zhou et al., Aug. 20, 2019, “Stent and Method of Inserting a Stent Into a Delivery Catheter”) discloses a stent for redirecting blood flow away from an aneurysmal sac. U.S. Pat. No. 10,398,441 (Warner et al., Sep. 3, 2019, “Vascular Occlusion”) discloses an occlusive system for the vasculature and an embolic coil used in such an occlusive system. U.S. Pat. No. 10,653,425 (Gorochow et al., May 19, 2020, “Layered Braided Aneurysm Treatment Device”) discloses a braided implant that can be secured within an aneurysm sac, occlude a majority of the aneurysm's neck, and at least partially fill the aneurysm sac. U.S. Pat. No. 10,716,574 (Lorenzo et al., Jul. 21, 2020, “Aneurysm Device and Delivery Method”) discloses a self-expanding braid for treating an aneurysm.

U.S. Pat. No. 10,905,430 (Lorenzo et al., Feb. 2, 2021, “Aneurysm Device and Delivery System”) discloses a braid for treating an aneurysm with a first radially expandable segment operable to move from a collapsed state within a microcatheter to a deployed state distal of the microcatheter and a second radially expandable segment operable to move from the collapsed state within the microcatheter to the deployed state distal of the microcatheter. U.S. Pat. No. 10,905,431 (Gorochow, Feb. 2, 2021, “Spiral Delivery System for Embolic Braid”) discloses a device for treating an aneurysm with a braided implant including a delivery tube having a spiral groove on an outer surface of the delivery tube and a braided implant having a spiral segment. U.S. Pat. No. 10,939,915 (Gorochow et al., Mar. 9, 2021, “Aneurysm Device and Delivery System”) discloses a braid for treating an aneurysm, wherein translating the braid causes a delivery portion to expand and form a distal sack as well as invert into itself.

U.S. patent application No. 20210137526 (Lee et al., May 13, 2021, “Embolic Devices for Occluding Body Lumens”) discloses an embolic device for placement in a body lumen, including: a first segment having a first linear configuration when located inside a catheter, the first segment being configured to form a first three-dimensional structure when outside the catheter, wherein the first three-dimensional structure defines a cavity; and a second segment extending from the first segment, the second segment having a second linear configuration when located inside the catheter, the second segment being configured to form a second three-dimensional structure when outside the catheter; wherein the cavity of the first three-dimensional structure is configured to accommodate at least a majority of the second three-dimensional structure.

U.S. Pat. No. 11,051,825 (Gorochow, Jul. 6, 2021, “Delivery System for Embolic Braid”) discloses a device for treating an aneurysm by releasing an implanted implant from a delivery system at a treatment site, including a braided implant attached to a releasing component that can be detachably engaged with a delivery tube and a pull wire. U.S. Pat. No. 11,058,430 (Gorochow et al., Jul. 13, 2021, “Aneurysm Device and Delivery System”) discloses a braid for treating an aneurysm, wherein the braid includes a proximal expandable portion for positioning inside the aneurysm and sealing across a neck of the aneurysm. U.S. Pat. No. 11,076,860 (Lorenzo, Aug. 3, 2021, “Aneurysm Occlusion Device”) discloses an occlusion device suitable for endovasculature treatment of an aneurysm in a blood vessel in a patient, including a substantially tubular structure having a proximal end region and a distal end region, having a first, expanded condition and a second, collapsed condition.

U.S. Pat. No. 11,076,861 (Gorochow et al., Aug. 3, 2021, “Folded Aneurysm Treatment Device and Delivery Method”) discloses an implant having a braided section that can be implanted in a deployed state such that, in the deployed state, the braided section folds to form an outer occlusive sack extending across a neck of an aneurysm. U.S. Pat. No. 11,278,292 (Gorochow et al., Mar. 22, 2022, “Inverting Braided Aneurysm Treatment System and Method”) discloses a tubular braid with an open end, a pinched end, and a predetermined shape.

### SUMMARY OF THE INVENTION

This invention is a device and method to occlude a cerebral aneurysm. This invention can be embodied in a device to occlude a cerebral aneurysm which includes: a longitudinal lumen (e.g. catheter) that is inserted into the parent blood vessel of an aneurysm; a flexible expandable member (such as a net or mesh) which is expanded within the aneurysm sack by the insertion of embolic members into that flexible expandable member; and a resilient expandable member (such as a cylindrical stent or ring stent) that is attached to a central portion of the flexible expandable member and expanded within the aneurysm sack in order to keep the flexible expandable member from slipping out of the aneurysm sack.

More specifically, this invention can be embodied in: (a) a longitudinal lumen (e.g. catheter) that is configured to be inserted into a blood vessel; (b) a flexible expandable member that is configured to travel through the longitudinal lumen, be inserted into an aneurysm, and then be expanded within the aneurysm sack; wherein this flexible expandable member is selected from the group consisting of a net, a mesh, a lattice, and a balloon with holes; wherein this flexible expandable member is sufficiently flexible to substantively conform to the contours of the walls of the aneurysm sack after the flexible expandable member is expanded within the aneurysm; (c) a resilient expandable member that is configured to travel through the longitudinal lumen, be inserted into the aneurysm, and then be expanded within the aneurysm sack; wherein this resilient expandable member resists contraction after it has been expanded; wherein a plane formed by the expanding circumference of this resilient expandable member is substantially parallel to the plane that centrally spans the circumference of the aneurysm neck; wherein a plane formed by the expanding circumference of this resilient expandable member spans the aneurysm sack at the sack's largest circumference parallel to the plane that centrally spans the circumference of the aneurysm neck; and wherein expansion of the resilient expandable member resiliently holds a central portion of the flexible expandable member against the walls of the aneurysm so that the flexible expandable member does not slip out of the aneurysm sack; and (d) a plurality of individual embolic members that are configured to travel through the longitudinal lumen, be inserted into the flexible expandable member within the aneurysm, and accumulate within the flexible expandable member; wherein the flexible expandable member does not allow the embolic members to escape out from the flexible expandable member; and wherein accumulation of the plurality of embolic members inside the flexible expandable member causes the flexible expandable member to expand.

### BRIEF INTRODUCTION TO THE FIGURES

FIGS. 1 through 3 show three sequential views (during deployment) of an embolic coil which forms interconnected contiguous loops within an aneurysm sack.

FIGS. 4 through 6 show three sequential views (during deployment) of an embolic coil with selective connection detachment for adjustable-size interconnected loops.

FIGS. 7 through 9 show three sequential views (during deployment) of a device and method to occlude an aneurysm which can be described as an embolic coil with selective connection formation for adjustable-size interconnected loops.

FIGS. 10 through 12 show three sequential views (during deployment) of a device and method to occlude an aneurysm which can be described as connecting and looping embolic members to occlude an aneurysm.

FIGS. 13 through 15 show three sequential views (during deployment) of a device and method to occlude an aneurysm which can be described as inserting and rotating embolic members to occlude an aneurysm.

FIGS. 16 through 18 show an example of a device and method to occlude an aneurysm which can be described as coil rotation to form a densely-packed mass within an aneurysm.

FIGS. 19 through 21 show three sequential views (during deployment) of a device and method to occlude an aneurysm which can be described as “string-of-pearls” rotation to form a densely-packed mass within an aneurysm.

FIGS. 22 through 24 show three sequential views (during deployment) of a device and method to occlude an aneurysm which can be described as aneurysm coil jailing using an expandable torus.

FIGS. 25 through 27 show three sequential views (during deployment) of a device and method to occlude an aneurysm which can be described as aneurysm occlusion using loops which are substantially-parallel to the aneurysm neck.

FIGS. 28 through 30 show three sequential views (during deployment) of a device and method to occlude an aneurysm which can be described as aneurysm occlusion using multiple centrally-aligned ellipsoids.

FIGS. 31 through 33 show three sequential views (during deployment) of a device and method to occlude an aneurysm which can be described as a “Saturn-shaped” device for aneurysm occlusion.

FIGS. 34 through 36 show three sequential views (during deployment) of a device and method to occlude an aneurysm which can be described as using concentric resilient and non-resilient intrasaccular members for aneurysm occlusion.

### DETAILED DESCRIPTION OF THE FIGURES

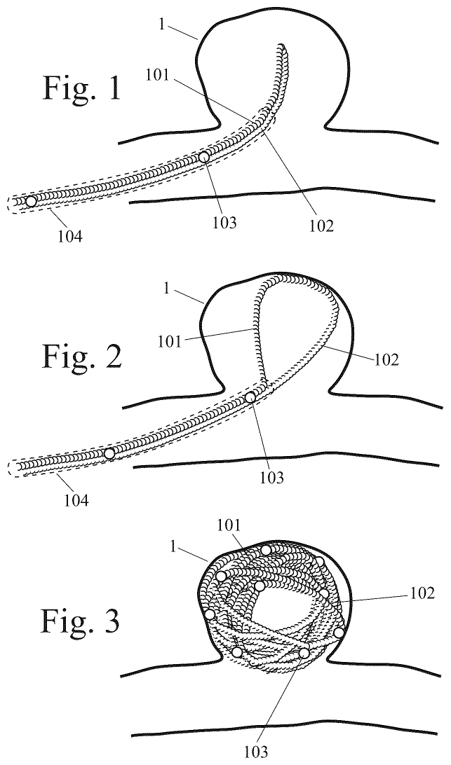

FIGS. 1 through 3 show an example of a device to occlude an aneurysm comprising: (a) a first longitudinal section of a flexible longitudinal embolic member that is configured to be inserted into an aneurysm; (b) a second longitudinal section of a flexible longitudinal embolic member that is configured to be inserted into the aneurysm, wherein the first and second longitudinal sections are connected and/or are contiguous sections of the same flexible longitudinal embolic member; (c) a plurality of connections between the first and second sections, wherein these connections connect the first and second longitudinal sections at a plurality of selected locations along their longitudinal axes; and (d) a longitudinal lumen (e.g. catheter) that is configured to be inserted into a blood vessel, wherein the first and second longitudinal sections travel through the lumen in order to be inserted into the aneurysm; wherein at least portions of the first and second longitudinal sections are configured in parallel within the lumen; wherein portions of the first and second longitudinal sections which are not connected by connections move apart from each other after exiting the lumen and connections move closer to each other after exiting the lumen in order to form a plurality of loops within the aneurysm; wherein part of the perimeter of a loop is comprised of a portion of the first longitudinal section and part of the perimeter of a loop is comprised of a portion of the second longitudinal section; wherein a loop has a contiguous 360-degree perimeter with ends which are connected to each other; and wherein loops are interconnected at the connections.

FIGS. 1 through 3 also show an example of a device to occlude an aneurysm comprising: (a) a longitudinal lumen (e.g. catheter) that is configured to be inserted into a blood vessel, wherein this blood vessel is the parent vessel from which an aneurysm has formed; (b) a first longitudinal flexible embolic member that is configured to travel through the longitudinal lumen and be inserted into the aneurysm sac; (c) a second longitudinal flexible embolic member that is configured to travel through the longitudinal lumen and be inserted into the aneurysm sac; wherein the longitudinal axes of the first and second embolic members are substantially parallel as these embolic members travel through the longitudinal lumen; and wherein the first and second embolic members are contiguous and/or connected to each other at their distal ends; and (d) a plurality of connections which connect the first and second embolic members at a plurality of locations along the lengths of the embolic members; wherein the segments of the first and second embolic members that are not connected by the connections move away from each other after they exit the longitudinal lumen, thereby forming loops within the aneurysm sac; wherein these loops are connected by the connections; and wherein accumulation of these loops within the aneurysm sac substantially occludes the aneurysm.

In an example, the longitudinal lumen (e.g. catheter) can be a removable catheter. In an example, the embolic members can be coils. In an example, the connections can connect the embolic members at uniformly-spaced locations along their lengths so as to form equal-size loops within the aneurysm sac and wherein these equal-size loops substantially span the circumference of the aneurysm sac. In an example, the connections can connect the embolic members at uniformly-spaced locations along their lengths so as to form equal-size loops within the aneurysm sac and these equal-size loops substantially span the circumference of the aneurysm sac without protruding into the parent vessel. In an example, the connections can connect the embolic members at non-uniformly-spaced locations along their lengths so as to form loops of different sizes within the aneurysm sac and these different size loops substantially occlude the interior as well as the circumference of the aneurysm sac.

We now discuss the specific components of FIGS. 1 through 3 in detail. FIGS. 1 through 3 show three sequential views of the same example of a device and method to occlude an aneurysm. To provide anatomical context, FIG. 1 shows a longitudinal cross-sectional view of an aneurysm sac 1 which has formed on a longitudinal blood vessel. FIG. 1 also shows an occlusive device comprising: a longitudinal lumen (e.g. catheter) 104 that has been inserted into the longitudinal blood vessel; a first longitudinal flexible embolic member 101 that travels through lumen 104 into aneurysm sac 1; a second longitudinal flexible embolic member 102 that travels through lumen 104 into aneurysm sac 1; and a plurality of connections (including 103) which connect first and second embolic members 101 and 102 at a plurality of locations along their longitudinal lengths. In this example, flexible embolic members 101 and 102 are two different segments (or sides) of the same continuous embolic member. In this example, this continuous member has two parallel segments or sides (comprising embolic members 101 and 102) within longitudinal lumen 104. In another example, embolic member 101 and embolic 102 can be different embolic members that are connected in some other manner at their distal ends.

In this example, flexible embolic members 101 and 102 are substantially parallel as they travel through longitudinal lumen (e.g. catheter) 104. However, as shown in FIG. 2, portions of embolic members 101 and 102 which are not connected to each other separate from each other after they exit longitudinal lumen 104 within aneurysm sac 1. In an example, this separation can be partly caused by pressure from contact with the wall of aneurysm sac 1. In an example, this separation can be partly caused by embolic members 101 and 102 having a shape memory with a shape that is restored after these embolic members exit longitudinal lumen 104. In the example that is shown in FIGS. 1 and 2, segments of embolic members 101 and 102 which are not connected by connections (such as 103) move away from each other after they exit longitudinal lumen 104.

FIG. 3 shows the accumulation of a plurality of interconnected, contiguous loops within aneurysm sac 1 as flexible longitudinal embolic members 101 and 102 continue to be pushed into aneurysm sac 1. These loops are pair-wise connected to each other by the plurality of connections (including connection 103). As shown in FIG. 3, accumulation of this plurality of loops within aneurysm sac 1 forms an embolic mass which substantially occludes the aneurysm. In this example, the interconnected and contiguous nature of these loops helps to prevent loops from prolapsing out of aneurysm sac 1 into the parent blood vessel. This can result in less prolapse of coils into the parent vessel than is the case with coils in the prior art which disperse and accumulate in a free-form spiraling manner within the aneurysm sac. Also, FIG. 3 shows longitudinal lumen (e.g. catheter) 104 as having been removed.

In the example shown in FIGS. 1 through 3, the connections (such as 103) between embolic members 101 and 102 are relatively evenly-spaced along the longitudinal lengths of embolic members 101 and 102. In an example, the spacing of these connections can be selected for a specific aneurysm with a specific size and shape in order to most efficiently occlude that specific aneurysm. In an example, the spacing of connections can differ between devices which are configured to occlude narrow-neck aneurysms and devices which are configured to occlude wide-neck aneurysms. In an example, the spacing of these connections can be pre-selected to vary along the length of embolic members 101 and 102 in order to most efficiently occlude an aneurysm at different times or stages during the occlusion procedure. For example, connections can be separated by longer distances at the most distal portions of embolic members 101 and 102 and become progressively shorter at more proximal portions of embolic members 101 and 102. In an example, the most distal connections can be spaced to form loops which span the entire circumference of the aneurysm sac but successive loops can become smaller and smaller to better fill the central space of the aneurysm sac. Relevant variations discussed elsewhere in this disclosure or in priority-linked disclosures can also be applied to this example.

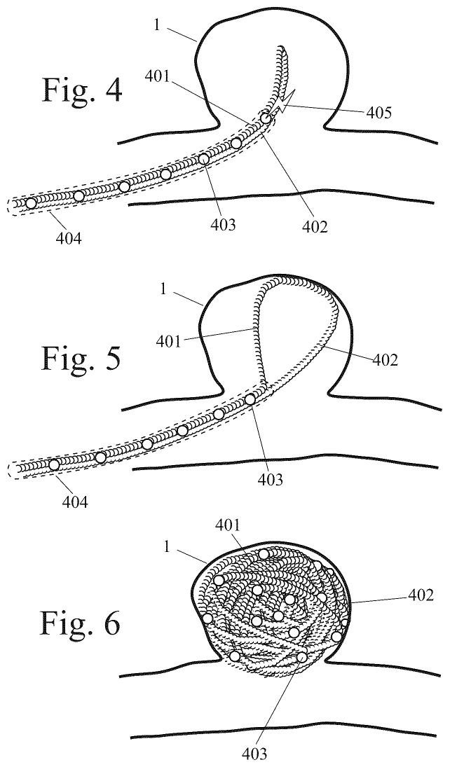

FIGS. 4 through 6 show an example of a device and method to occlude an aneurysm which can be described as an embolic coil with selective connection detachment for adjustable-size interconnected loops. More specifically, FIGS. 4 through 6 show an example of a device to occlude an aneurysm comprising: (a) a first longitudinal section of a flexible longitudinal embolic member that is configured to be inserted into an aneurysm; (b) a second longitudinal section of a flexible longitudinal embolic member that is configured to be inserted into the aneurysm, wherein the first and second longitudinal sections are connected and/or are contiguous sections of the same flexible longitudinal embolic member; (c) selectively-detachable connections between the first and second sections, wherein these selectively-detachable connections connect the first and second longitudinal sections at a plurality of selected locations along their longitudinal axes, and wherein a subset of the selectively-detachable connections are selectively detached during insertion of the first and second longitudinal sections into the aneurysm; and (d) a longitudinal lumen (e.g. catheter) that is configured to be inserted into a blood vessel, wherein the first and second longitudinal sections travel through the lumen in order to be inserted into the aneurysm; wherein at least a portion of the first and second longitudinal sections are substantially parallel to each other within the lumen; wherein portions of the first and second longitudinal sections which are not connected by selectively-detachable connections move apart from each other after exiting the lumen and selectively-detachable connections which are not detached move closer to each other after exiting the lumen in order to form a plurality of loops within the aneurysm; wherein part of the perimeter of a loop is comprised of a portion of the first longitudinal section and part of the perimeter of a loop is comprised of a portion of the second longitudinal section; wherein a loop has a contiguous 360-degree perimeter with ends which are connected to each other; wherein loops are interconnected at the selectively-detachable connections which are not detached; and wherein the loop size of at least one loop is selectively determined by selective detachment of a subset of the selectively-detachable connections during insertion of the first and second longitudinal sections.

FIGS. 4 through 6 also show an example of a device to occlude an aneurysm comprising: (a) a longitudinal lumen (e.g. catheter) that is configured to be inserted into a blood vessel, wherein this blood vessel is the parent vessel from which an aneurysm has formed; (b) a first longitudinal flexible embolic member that is configured to travel through the longitudinal lumen and be inserted into the aneurysm sac; (c) a second longitudinal flexible embolic member that is configured to travel through the longitudinal lumen and be inserted into the aneurysm sac; wherein the longitudinal axes of the first and second embolic members are substantially parallel as these embolic members travel through the longitudinal lumen; and wherein the first and second embolic members are contiguous and/or connected to each other at their distal ends; and (d) a plurality of disconnectable connections which connect the first and second embolic members at a plurality of locations along the lengths of the embolic members; wherein these a subset of these connections can be selected and disconnected during the insertion of the longitudinal members; wherein the segments of the first and second embolic members that are not connected by the remaining connections move away from each other after they exit the longitudinal lumen, thereby forming loops within the aneurysm sac; wherein these loops are connected by the remaining connections; and wherein accumulation of these loops within the aneurysm sac substantially occludes the aneurysm.

In an example, the longitudinal lumen can be a removable catheter. In an example, the embolic members can be coils. In an example, a subset of the connections can be disconnected by application of electric current. In an example, a subset of the connections can be disconnected by a cutting mechanism. In an example, the selective disconnection of a subset of connections can cause loops of different sizes and different size loops occlude a greater volume of the aneurysm sac than same size loops. In an example, the selective disconnection of a subset of connections during implantation of the device can enable custom-fitting different size loops to the size and shape of the aneurysm.

We now discuss the specific components of FIGS. 4 through 6 in detail. FIGS. 4 through 6 show three sequential views of the same example of a device and method to occlude an aneurysm. FIG. 4 shows an occlusive device comprising: a longitudinal lumen (e.g. catheter) 404 that has been inserted into the longitudinal blood vessel; a first longitudinal flexible embolic member 401 that travels through lumen 404 into aneurysm sac 1; a second longitudinal flexible embolic member 402 that travels through lumen 404 into aneurysm sac 1; and a plurality of disconnectable and/or detachable connections (including 403) which connect first and second embolic members 401 and 402 at a plurality of locations along their longitudinal lengths. In this example, flexible embolic members 401 and 402 are two different segments (or sides) of the same continuous embolic member. In this example, this continuous member has two parallel segments or sides (comprising embolic members 401 and 402) within longitudinal lumen 404. In another example, embolic member 401 and embolic 402 can be different embolic members that are connected in some other manner at their distal ends.

FIG. 4 shows the disconnection and/or detachment of one of the disconnectable and/or detachable connections during the implantable procedure. In this example, this disconnection and/or detachment is done by means of an electric current 405 which melts one of the connections. In this example, connection 403 is among the connections which are not disconnected and/or detached. As shown in FIG. 5, portions of embolic members 401 and 402 which are not connected to each other separate from each other after they exit longitudinal lumen (e.g. catheter) 404 within aneurysm sac 1. Selective disconnection and/or detachment of a subset of the plurality of connections between embolic members 401 and 402 during the implantation procedure can enable the formation of different size loops within the aneurysm sac so as to most efficiently occlude the aneurysm sac.

In an example, the selection of which connections to disconnect and/or detach can be done by a user, in real time, during the implantation procedure. This selection can be informed by real-time imaging concerning the placement of the longitudinal lumen (e.g. catheter) with respect to the aneurysm sac and/or real-time imaging concerning the progressive occlusion of the aneurysm sac. Enabling a user to selectively disconnect and/or detach selected connections during a procedure can avoid having to stock a variety of occluding members with a variety of connection spacing parameters. A standard occluding device with a standard set of connections can be modified by a user, in real time during the implantation procedure, in order to create different size loops in a specific sequence for an aneurysm's specific size and shape.

FIG. 6 shows the accumulation of a plurality of interconnected, contiguous loops within aneurysm sac 1 as some of the connections are disconnected and as flexible longitudinal embolic members 401 and 402 continue to be pushed into aneurysm sac 1. These loops are pair-wise connected to each other by the plurality of remaining connections (including connection 403). As shown in FIG. 3, accumulation of this plurality of loops within aneurysm sac 1 forms an embolic mass which substantially occludes the aneurysm. The interconnected and contiguous nature of these loops can help to reduce the probability of a loop prolapsing out of the aneurysm sac as compared to coils in the prior art which accumulate within the aneurysm sac in a less-controlled spiraling manner. Also, FIG. 6 shows longitudinal lumen (e.g. catheter) 404 as having been removed. Relevant variations discussed elsewhere in this disclosure or in priority-linked disclosures can also be applied to this example.

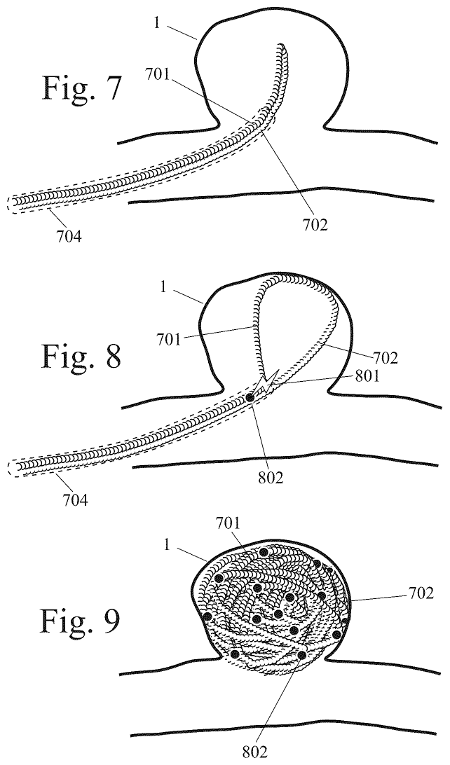

FIGS. 7 through 9 show an example of a device and method to occlude an aneurysm which can be described as an embolic coil with selective connection formation for adjustable-size interconnected loops. More specifically, FIGS. 7 through 9 show an example of a device to occlude an aneurysm comprising: (a) a first longitudinal section of a flexible longitudinal embolic member that is configured to be inserted into an aneurysm; (b) a second longitudinal section of a flexible longitudinal embolic member that is configured to be inserted into the aneurysm, wherein the first and second longitudinal sections are connected and/or are contiguous sections of the same flexible longitudinal embolic member; (c) selectively-formed connections between the first and second sections, wherein these selectively-formed connections connect the first and second longitudinal sections at a plurality of selected locations along their longitudinal axes, and wherein these selectively-formed connections are selectively formed during insertion of the first and second longitudinal sections into the aneurysm; and (d) a longitudinal lumen (e.g. catheter) that is configured to be inserted into a blood vessel, wherein the first and second longitudinal sections travel through the lumen in order to be inserted into the aneurysm; wherein at least a portion of the first and second longitudinal sections are substantially parallel to each other within the lumen; wherein portions of the first and second longitudinal sections which are not connected by selectively-formed connections move apart from each other after exiting the lumen and selectively-formed connections move closer to each other after exiting the lumen in order to form a plurality of loops within the aneurysm; wherein part of the perimeter of a loop is comprised of a portion of the first longitudinal section and part of the perimeter of a loop is comprised of a portion of the second longitudinal section; wherein a loop has a contiguous 360-degree perimeter with ends which are connected to each other; wherein loops are interconnected at the selectively-formed connections; and wherein the loop size of at least one loop is selectively determined by selective formation of a selectively-formed connection during insertion of the first and second longitudinal sections.

FIGS. 7 through 9 also show an example of a device to occlude an aneurysm comprising: (a) a longitudinal lumen (e.g. catheter) that is configured to be inserted into a blood vessel, wherein this blood vessel is the parent vessel from which an aneurysm has formed; (b) a first longitudinal flexible embolic member that is configured to travel through the longitudinal lumen and be inserted into the aneurysm sac; (c) a second longitudinal flexible embolic member that is configured to travel through the longitudinal lumen and be inserted into the aneurysm sac; wherein the longitudinal axes of the first and second embolic members are substantially parallel as these embolic members travel through the longitudinal lumen; and wherein the first and second embolic members are contiguous and/or connected to each other at their distal ends; and (d) a plurality of connections between the first and second embolic members which are formed to connect the first and second embolic members at a plurality of locations along the lengths of the embolic members; wherein these connections are formed during the insertion of the longitudinal members; wherein the segments of the first and second embolic members that are not connected by the connections move away from each other after they exit the longitudinal lumen, thereby forming loops within the aneurysm sac; and wherein accumulation of these loops within the aneurysm sac substantially occludes the aneurysm.

In an example, the longitudinal lumen (e.g. catheter) can be a removable catheter. In an example, the embolic members can be coils. In an example, the connections between the first and second embolic members can be formed by application of electric current. In an example, the connections between the first and second embolic members can be formed by compression, linking, stapling, snapping, latching, or adhesion. In an example, the selective formation of connections between the first and second embolic members can cause loops of different sizes and using different size loops can occlude a greater volume of the aneurysm sac than using same size loops. In an example, the selective formation of connections between the first and second embolic members during implantation of the device can enable custom-fitting different size loops to the size and shape of the aneurysm.

We now discuss the specific components of FIGS. 7 through 9 in detail. FIG. 7 shows an occlusive device comprising: a longitudinal lumen (e.g. catheter) 704 that has been inserted into a longitudinal blood vessel from which an aneurysm has formed; a first longitudinal flexible embolic member 701 that travels through lumen 704 into aneurysm sac 1; and a second longitudinal flexible embolic member 702 that travels through lumen 704 into aneurysm sac 1. In this example, flexible embolic members 701 and 702 are two different segments (or sides) of the same continuous embolic member.

FIG. 8 shows the formation of a connection 802 between embolic members 701 and 702 by application of electric current 801 during the implantable procedure. In other examples, connections between embolic members 701 and 702 can be formed by a means selected from the group consisting of: adhesion, fusing, compressing, pinching, stapling, snapping, linking, twisting, binding, and tying. As shown in FIG. 8, portions of embolic members 701 and 702 which are not connected to each other separate from each other after they exit longitudinal lumen (e.g. catheter) 704 within aneurysm sac 1. Selective formation of connections (such as 802) between embolic members 701 and 702 during the implantation procedure enables the formation of different size loops within the aneurysm sac so as to most efficiently occlude the aneurysm sac.

In an example, the selection of where to form connections along the lengths of embolic members 701 and 702 can be done by a user, in real time, during the implantation procedure. This selection can be informed by real-time imaging concerning the placement of the longitudinal lumen (e.g. catheter) with respect to the aneurysm sac and/or real-time imaging concerning the progressive occlusion of the aneurysm sac. Enabling a user to selectively form connections during a procedure can avoid having to stock a variety of occluding members with a variety of connection spacing parameters. A standard occluding device can be modified by a user, in real time during the implantation procedure, in order to create different size loops in a specific sequence for an aneurysm's specific size and shape.

FIG. 9 shows the accumulation of a plurality of interconnected, contiguous loops within aneurysm sac 1 as connections are formed and flexible longitudinal embolic members 701 and 702 are pushed into aneurysm sac 1. These loops are pair-wise connected to each other by the plurality of connections (including connection 802). The interconnected and contiguous nature of these loops can help to reduce the probability of a loop prolapsing out of the aneurysm sac as compared to coils in the prior art which accumulate within the aneurysm sac in a less-controlled spiraling manner. Also, FIG. 9 shows longitudinal lumen (e.g. catheter) 704 as having been removed. Relevant variations discussed elsewhere in this disclosure or in priority-linked disclosures can also be applied to this example.

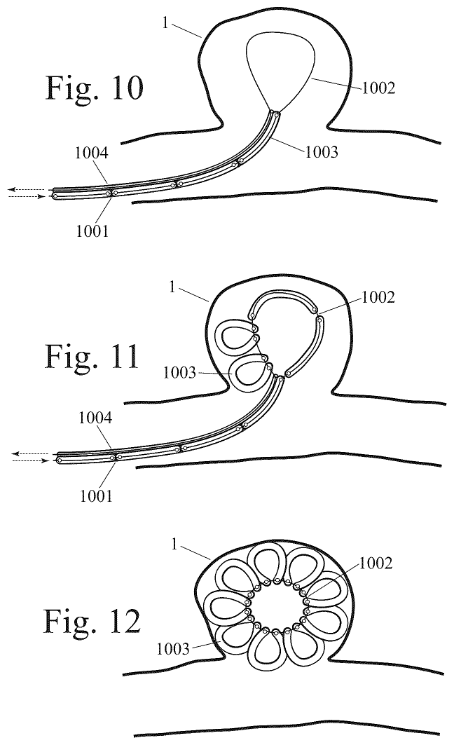

FIGS. 10 through 12 show an example of a device and method to occlude an aneurysm which can be described as connecting and looping embolic members to occlude an aneurysm. More specifically, FIGS. 10 through 12 show an example of a device to occlude an aneurysm comprising: (a) a longitudinal lumen (e.g. catheter) that is configured to be inserted into a blood vessel; (b) a connecting member that travels through the lumen and is configured to be inserted into an aneurysm; wherein this connecting member is longitudinal and flexible; wherein an embolic member has a longitudinal axis that spans from its proximal end to its distal end as it travels through the lumen and a cross-sectional area that is substantively perpendicular to its longitudinal axis; (c) a plurality of embolic members that travel in a series configuration through the lumen and are configured to be inserted into the aneurysm; wherein these embolic members are longitudinal and flexible; wherein these embolic members are connected and/or linked to each other by the connecting member; wherein an embolic member has a longitudinal axis that spans from its proximal end to its distal end as it travels through the lumen and a cross-sectional area that is substantively perpendicular to its longitudinal axis; wherein the longitudinally-central portion of an embolic member bends away from the connecting member after the embolic member exits the lumen due to the two ends of the embolic member being pulled or pushed towards each other; wherein this bending of the longitudinally-central portion of an embolic member causes the embolic member to form a loop within the aneurysm; and wherein the formation of multiple loops within the aneurysm helps to embolize the aneurysm.

FIGS. 10 through 12 also show an example of a device to occlude an aneurysm comprising: (a) a longitudinal lumen (e.g. catheter) that is configured to be inserted into a blood vessel, wherein this blood vessel is the parent vessel from which an aneurysm has formed; (b) a longitudinal flexible member that is configured to travel through the longitudinal lumen and be inserted into the aneurysm sac; (c) a plurality of flexible embolic members that are configured to travel through the longitudinal lumen and be inserted into the aneurysm sac; wherein these flexible embolic members are connected by the longitudinal flexible member; wherein the ends of a flexible embolic member are separated by a first distance as the embolic member travels through the longitudinal lumen; wherein the ends of this flexible embolic member are separated by a second distance after the embolic member exits the longitudinal lumen; wherein the second distance is less than the first distance; and wherein the accumulation of flexible embolic members in the aneurysm sac substantively occludes the aneurysm sac.

In an example, the longitudinal lumen (e.g. catheter) can be a removable catheter. In an example, the longitudinal flexible member can be selected from the group consisting of: wire, string, filament, chain, and coil. In an example, an embolic member can have a longitudinal axis, wherein this longitudinal axis is substantively straight as the embolic member travels through the longitudinal lumen, and wherein this longitudinal axis becomes arcuate after the embolic member exits the longitudinal lumen. In an example, the ends of a flexible embolic member can move closer together after the embolic member exits the longitudinal lumen because the embolic member comprises a material with a shape memory. In an example, the ends of a flexible embolic member can move closer together after the embolic member exits the longitudinal lumen because the embolic member is compressed by the force of other embolic members in the aneurysm sac. In an example, the ends of a flexible embolic member can be slidably connected to the longitudinal flexible member.

We now discuss the specific components of the example that is shown FIGS. 10 through 12 in detail. FIG. 10 shows an example of an occlusive device comprising: a first longitudinal lumen (e.g. catheter) 1001 that is configured to be inserted into a blood vessel, wherein this blood vessel is the parent vessel from which an aneurysm has formed; a second longitudinal lumen 1004 that is configured to be inserted into the blood vessel; a longitudinal flexible member 1002 that is configured to travel through one of the longitudinal lumens and to be inserted into the aneurysm sac; and a plurality of flexible embolic members, including 1003, that are configured to travel through lumen 1001 and be inserted into the aneurysm sac. In this example, flexible member 1002 comprises a loop wherein one side of the loop travels through lumen 1001 and the other side travels through lumen 1004. In another example, a device can only have one lumen and both sides of a loop can travel through this one lumen. Also, as shown in FIG. 10, the flexible embolic members (including 1003) can be connected by longitudinal flexible member 1002.

As shown in FIG. 11, the ends of a flexible embolic member (including 1003) can be separated by a first distance as the embolic member travels through lumen (e.g. catheter) 1001 and the ends of the flexible embolic member are separated by a second distance after the embolic member exits lumen 1001. The second distance is less than the first distance. In this example, the central axis of an embolic member (such as 1003) can change from being relatively straight within lumen 1001 to being relatively arcuate after it exits lumen 1001.

In an example, flexible embolic members (such as 1003) can be relatively straight as they travel through lumen (e.g. catheter) 1001, but they can curve into loops after exiting lumen 1001. In this example, the accumulation of curved, looping embolic members in aneurysm sac 1 forms a looping embolic mass that substantively occludes the aneurysm sac. In this example, the loops look similar to flower petals as they extend outwards from a central circle that is formed by longitudinal flexible member 1002. In this example, this embolic “flower petal” arrangement has a radial-expansion plane which is substantially perpendicular to the plane formed by the central circumference of the aneurysm neck. In an example, an embolic “flower petal” arrangement can have a radial-expansion plane which is substantially parallel to the plane formed by the central circumference of the aneurysm neck.

In an example, both ends of flexible embolic members (such as 1003) can be slidably attached to longitudinal flexible member 1002. In an example, pressure from other flexible embolic members accumulating in the aneurysm sac can push the ends of flexible embolic members (such as 1003) together as they accumulate in the aneurysm sac. In an example, one end of a flexible embolic member (such as 1003) can be slidably attached to longitudinal flexible member 1002, but the other end of the flexible embolic member may not be attached to the longitudinal flexible member.

In an example, the distal portion of longitudinal flexible member 1002 can be fused into a circle and detached from the proximal portion of longitudinal flexible member 1002 after a sufficient number of loops have accumulated in the aneurysm sac to occlude the aneurysm. FIG. 12 shows the distal portion of longitudinal flexible member 1002 having been fused into a circle and detached. FIG. 12 also shows lumen (e.g. catheter) s 1001 and 1004 having been removed. Relevant variations discussed elsewhere in this disclosure or in priority-linked disclosures can also be applied to this example.

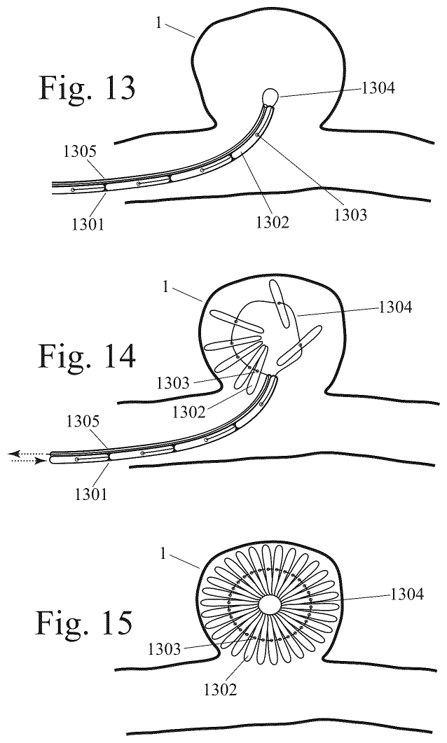

FIGS. 13 through 15 show an example of a device and method to occlude an aneurysm which can be described as inserting and rotating embolic members to occlude an aneurysm. More specifically, FIGS. 13 through 15 show an example of a device to occlude an aneurysm comprising: (a) a longitudinal lumen (e.g. catheter) that is configured to be inserted into a blood vessel; (b) a connecting member that travels through the lumen and is configured to be inserted into an aneurysm; wherein this connecting member is longitudinal and flexible; wherein this connecting member has a longitudinal axis from a proximal end to a distal end as it travels through the lumen and has a cross-sectional area that is substantially perpendicular to its longitudinal axis; and (c) a plurality of embolic members that travel in a series configuration through the lumen and are configured to be inserted into the aneurysm; wherein these embolic members are connected and/or linked to each other by the connecting member; wherein an embolic member has a longitudinal axis that spans from its proximal end to its distal end as it travels through the lumen and a cross-sectional area that is substantively perpendicular to its longitudinal axis; wherein the longitudinal axis of the embolic member has a first orientation with respect to the longitudinal axis of the connecting member as the embolic member travels within the lumen; wherein the longitudinal axis of the embolic member rotates to a second orientation with respect to the longitudinal axis of the connecting member after the embolic member exits the lumen; wherein the second orientation is different than the first orientation; and wherein this second orientation of the longitudinal axis of the embolic member is substantially perpendicular to the longitudinal axis of the connecting member in the vicinity of the embolic member.

FIGS. 13 through 15 also show an example of a device to occlude an aneurysm comprising: (a) a longitudinal lumen (e.g. catheter) that is configured to be inserted into a blood vessel, wherein this blood vessel is the parent vessel from which an aneurysm has formed; (b) a longitudinal flexible member that is configured to travel through the longitudinal lumen and be inserted into aneurysm sac; and (c) a plurality of longitudinal embolic members that are configured to travel through the longitudinal lumen and be inserted into the aneurysm sac; wherein these embolic members are connected by the longitudinal flexible member; wherein the longitudinal axis of an embolic member has a first orientation with respect to the longitudinal axis of the longitudinal flexible member as this embolic member travels through the longitudinal lumen; wherein the longitudinal axis of this embolic member has a second orientation with respect to the longitudinal axis of the longitudinal flexible member after this embolic member exits the longitudinal lumen; wherein the second orientation is closer to perpendicular than the first orientation; and wherein the accumulation of embolic members in the aneurysm sac substantively occludes the aneurysm sac.

In an example, the longitudinal lumen (e.g. catheter) can be a removable catheter. In an example, the longitudinal flexible member can be selected from the group consisting of: wire, string, filament, chain, and coil. In an example, the longitudinal axis of an embolic member can be substantively parallel to the longitudinal axis of the longitudinal flexible member as the embolic member travels through the longitudinal lumen and the longitudinal axis of an embolic member can become substantively perpendicular to the longitudinal axis of the longitudinal flexible member after the embolic member exits the longitudinal lumen. In an example, the longitudinal flexible member can slide through central portions of the embolic members.

We now discuss the specific components of FIGS. 13 through 15 in detail. FIG. 13 shows an example of an occlusive device comprising: a first longitudinal lumen (e.g. catheter) 1301 that is configured to be inserted into a blood vessel from which an aneurysm has formed; a second longitudinal lumen 1305 that is configured to be inserted into the blood vessel; a longitudinal flexible member 1304 that is configured to travel through one or both of these longitudinal lumens and to be inserted into the aneurysm sac; and a plurality of flexible embolic members (such as 1302) that are configured to travel through lumen 1301 and be inserted into the aneurysm sac, wherein each of these embolic members has a central opening (such as 1303) through which it is connected to longitudinal flexible member 1304. In this example, flexible member 1304 comprises a loop wherein one side of the loop travels through lumen 1301 and the other side travels through lumen 1305. In another example, a device can only have one lumen and both sides of a loop can travel through this one lumen.

In this example, the plurality of embolic members (including 1302) are connected by longitudinal flexible member 1304. In this example, the embolic members are slidably connected to the longitudinal flexible member by having the longitudinal flexible member go through openings (such as 1303) in the central portions of the embolic members. In this example, the longitudinal axis of an embolic member (such as 1302) has a first orientation with respect to the longitudinal axis of longitudinal flexible member 1304 as this embolic member travels through lumen (e.g. catheter) 1305 and a second orientation with respect to the longitudinal axis of longitudinal flexible member 1304 after this embolic member exits lumen 1305. In an example, the second orientation can be closer to perpendicular than the first orientation.

As was the case with previous examples, the accumulation of embolic members in the aneurysm sac substantively occludes the aneurysm sac. In an example, the distal portion of longitudinal flexible member 1304 can be fused into a circle and detached from the proximal portion of longitudinal flexible member 1304 after a sufficient number of embolic members have accumulated in the aneurysm sac to occlude the aneurysm. FIG. 15 shows the distal portion of longitudinal flexible member 1304 having been fused into a circle and detached. FIG. 15 also shows lumen (e.g. catheter) s 1301 and 1305 having been removed. Relevant variations discussed elsewhere in this disclosure or in priority-linked disclosures can also be applied to this example.

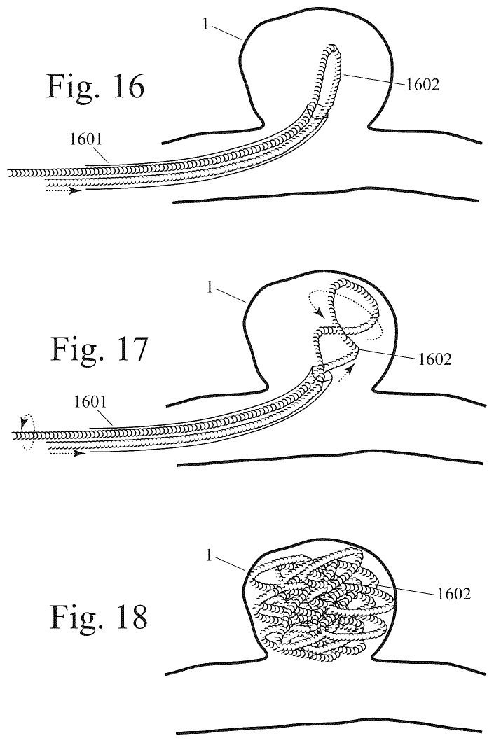

FIGS. 16 through 18 show an example of a device and method to occlude an aneurysm which can be described as coil rotation to form a densely-packed mass within an aneurysm. More specifically, FIGS. 16 through 18 show an example of a device to occlude an aneurysm comprising: (a) a first longitudinal section of a flexible longitudinal embolic member that travels through a lumen (e.g. catheter) that is configured to be inserted into a blood vessel; wherein this embolic member is configured to be inserted into an aneurysm; and (b) a second longitudinal section of a flexible longitudinal embolic member that travels through a lumen that is configured to be inserted into a blood vessel; wherein this embolic member is configured to be inserted into an aneurysm; wherein this second longitudinal section is distally connected to the first longitudinal section; wherein the first longitudinal section is rotated around its longitudinal axis as the second longitudinal section is pushed from the lumen into the aneurysm; and wherein the combination of pushing the second section into the aneurysm and rotating the first section causes the first and second sections to become wound around each other and thereby create a densely-packed mass within the aneurysm.

FIGS. 16 through 18 also show an example of a device to occlude an aneurysm comprising: (a) a longitudinal lumen (e.g. catheter) that is configured to be inserted into a blood vessel, wherein this blood vessel is the parent vessel from which an aneurysm has formed; (b) a first longitudinal flexible embolic member that is configured to travel through the longitudinal lumen and be inserted into the aneurysm sac; and (c) a second longitudinal flexible embolic member that is configured to travel through the longitudinal lumen and be inserted into the aneurysm sac; wherein the first and second embolic members are contiguous and/or connected to each other at their distal ends; and wherein the first embolic member is rotated around its longitudinal axis as the second embolic member is inserted into the aneurysm sac, causing the first and second embolic members to entwine around each other within the aneurysm sac.

In an example, the embolic members can be coils. In an example, both the first and second embolic members can be inserted into an aneurysm sac while the first embolic member is being rotated. In an example, the rate at which a first embolic member and/or a second embolic member is inserted into the aneurysm sac, the rate at which the first embolic member is rotated, or both of these rates can be selected based on the size and shape of an aneurysm sac so that the resulting entwined members substantially occlude the aneurysm sac. In an example, the rate at which a first embolic member and/or a second embolic member is inserted into the aneurysm sac, the rate at which a first embolic member is rotated, or both of these rates can be varied during implantation of the device so that the resulting entwined members substantially occlude an aneurysm sac. In an example, the rate at which the first embolic member is rotated can be adjusted from a faster rate to a slower rate during implantation.

FIGS. 16 through 18 also show an example of a device to occlude an aneurysm comprising: (a) a longitudinal lumen (e.g. catheter) that is configured to be inserted into a blood vessel, wherein this blood vessel is the parent vessel from which an aneurysm has formed; and (b) a longitudinal flexible embolic loop that is configured to travel through the longitudinal lumen and be inserted into the aneurysm sac; wherein a first strand (or side) of the embolic loop is rotated around its longitudinal axis while a second strand (or side) of the embolic loop is pushed into the aneurysm; and wherein the combination of rotating the first strand (or side) of the loop while pushing the second strand (or side) of the loop into the aneurysm causes the first and second strands (or sides) to entwine around each other within the aneurysm sac.

In an example, both the first and second strands (or sides) of the loop can be inserted into the aneurysm sac while the first strand (or side) of the loop is being rotated. In an example, the rate at which the first strand (or side) of the loop and/or second strand (or side) of the loop is inserted into the aneurysm sac, the rate at which the first strand (or side) of the loop is rotated, or both of these rates can be selected based on the size and shape of an aneurysm sac so that the resulting entwined mass substantially occludes the aneurysm sac. In an example, one or both of these rates can be varied during implantation of the device. In an example, the rate at which the first strand (or side) of the loop is rotated can be varied from a faster rate to a slower rate during the implantation.

FIGS. 16 through 18 also show an example of a method for occluding an aneurysm comprising: (a) inserting a first longitudinal section of a flexible longitudinal embolic member through a lumen (e.g. catheter) and into an aneurysm; and (b) rotating this first longitudinal section of a flexible longitudinal embolic member around its longitudinal axis as a second longitudinal section is pushed into the aneurysm; wherein this second longitudinal section is distally connected to the first longitudinal section; and wherein the combination of pushing the second section into the aneurysm and rotating the first section causes the first and second sections to become wound around each other and thereby create a densely-packed mass within the aneurysm.

We now discuss the specific components of FIGS. 16 through 18 in more detail. FIG. 16 shows an occlusive device that comprises: longitudinal lumen (e.g. catheter) 1601 that is configured to be inserted into a blood vessel from which an aneurysm has formed; and longitudinal flexible embolic loop 1602 that is configured to travel through the longitudinal lumen and be inserted into the aneurysm sac. In this example, a first strand (or side) of embolic loop 1602 is rotated around its longitudinal axis while a second strand (or side) of embolic loop 1602 is pushed into the aneurysm. As shown in FIG. 17, the combination of rotating the first strand (or side) of loop 1602 around its longitudinal axis while pushing the second strand (or side) of loop 1602 into the aneurysm causes the first and second strands (or sides) to wind around each other within the aneurysm sac. As shown in FIG. 18, this creates an entwined mass which occludes the aneurysm sac.

In an example, the rate at which the first strand of loop 1602 is rotated, the rate at which one or both strands of loop 1602 are inserted into the aneurysm sac, or both rates can be selected based on the size and shape of an aneurysm sac. In an example, one or both of these rates can be varied during implantation. In an example, the rate at which the first strand of loop 1602 is rotated can vary from a faster rate to a slower rate during implantation. In an example, the rate at which the second strand of loop 1602 is pushed into the aneurysm sac can vary from a slower rate to a faster rate during implantation. Relevant variations discussed elsewhere in this disclosure or in priority-linked disclosures can also be applied to this example.

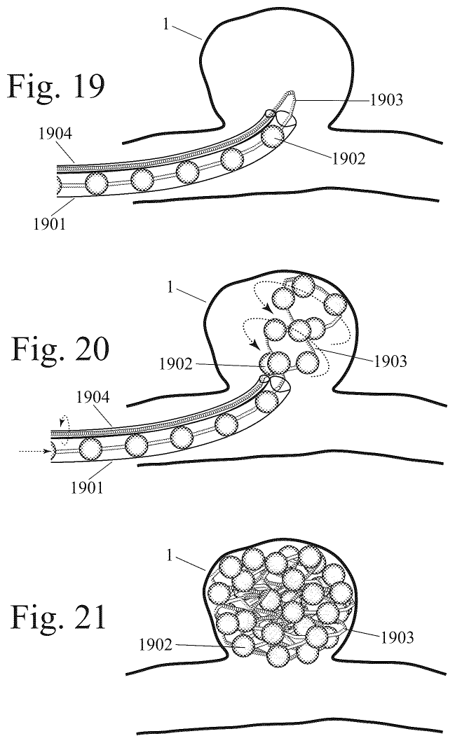

FIGS. 19 through 21 show an example of a device and method to occlude an aneurysm which can be described as “string-of-pearls” rotation to form a densely-packed mass within an aneurysm. More specifically, FIGS. 19 through 21 show an example of a device to occlude an aneurysm comprising: (a) a first longitudinal section of a flexible longitudinal embolic member that travels through a lumen (e.g. catheter) that is configured to be inserted into a blood vessel; wherein this embolic member is configured to be inserted into an aneurysm; (b) a second longitudinal section of a flexible longitudinal embolic member that travels through a lumen that is configured to be inserted into a blood vessel; wherein this embolic member is configured to be inserted into an aneurysm; wherein this second longitudinal section is configured to be pushed from the lumen into the aneurysm; and wherein this second longitudinal section comprises a series of separate embolic members which are linked by a thin flexible central member; wherein this second longitudinal section is distally connected to the first longitudinal section; wherein the first longitudinal section is rotated around its longitudinal axis as the second longitudinal section is pushed from the lumen into the aneurysm; and wherein the combination of pushing the second section into the aneurysm and rotating the first section causes the first and second sections to become wound around each other and thereby create a densely-packed mass within the aneurysm.

FIGS. 19 through 21 also show an example of a device to occlude an aneurysm comprising: (a) a longitudinal lumen (e.g. catheter) that is configured to be inserted into a blood vessel, wherein this blood vessel is the parent vessel from which an aneurysm has formed; (b) a first longitudinal flexible embolic member that is configured to travel through the longitudinal lumen and be inserted into the aneurysm sac; (c) a second longitudinal flexible embolic member that is configured to travel through the longitudinal lumen and be inserted into the aneurysm sac; wherein the first and second embolic members are contiguous and/or connected to each other at their distal ends; and wherein the first embolic member is rotated around its longitudinal axis as the second embolic member is inserted into the aneurysm sac, causing the first and second embolic members to entwine around each other within the aneurysm sac; and (d) a series of embolic members which are configured to travel through the longitudinal lumen and be inserted into the aneurysm sac; wherein these embolic members are connected by the first flexible member, the second flexible member, or both the first and second flexible members.

In an example, the longitudinal lumen (e.g. catheter) can be a removable catheter. In an example, the longitudinal flexible member can be selected from the group consisting of: wire, string, filament, chain, and coil. In an example, the rate at which the first flexible member and/or the second flexible member is inserted into the aneurysm sac, the rate at which the first flexible member is rotated, or both of these rates can be selected based on the size and shape of the aneurysm sac so that the resulting mass substantially occludes the aneurysm sac. In an example, the rate at which the first flexible member and/or the second flexible member is inserted into the aneurysm sac, the rate at which the first flexible member is rotated, or both of these rates can be varied during implantation of the device so that the resulting mass substantially occludes the aneurysm sac. In an example, the rate at which the first flexible member is rotated can be varied from a faster rate to a slower rate during the implantation of the device. In an example, the plurality of embolic members can be evenly-spaced along the longitudinal axis of the first flexible member, the second flexible member, or both the first and second flexible members. In an example, the plurality of embolic members can be unevenly-spaced along the longitudinal axis of the first flexible member, the second flexible member, or both the first and second flexible members and wherein the uneven spacing of the embolic members is selected based on the size and shape of the aneurysm.

FIGS. 19 through 21 also show an example of a device to occlude an aneurysm comprising: (a) a first longitudinal lumen (e.g. catheter) that is configured to be inserted into a blood vessel that is the parent vessel from which an aneurysm has formed; (b) a second longitudinal lumen that is configured to be inserted into the blood vessel; (c) a flexible loop that is configured to travel through the first and/or second longitudinal lumens and be inserted into the aneurysm sac, wherein a first strand (or side) of the loop is rotated around its longitudinal axis while a second strand (or side) of the loop is inserted into the aneurysm sac; and (d) a series of embolic members which are configured to travel through the longitudinal lumen and be inserted into the aneurysm sac; wherein these embolic members are connected by the second strand (or side) of the flexible loop.

In an example, rotation of a first strand (or side) of the flexible loop combined with insertion of a second strand (or side) of the loop (including attached embolic members) into the aneurysm sac can create a entwined embolic mass within the aneurysm sac. In an example, a first strand (or side) of the flexible loop can travel through a first longitudinal lumen (e.g. catheter) and a second strand (or side) of the flexible loop can travel through a second longitudinal lumen. In another example, a device may have only one longitudinal lumen and both strands (or sides) of the flexible loop can travel through the same longitudinal lumen.

FIGS. 19 through 21 also show an example of a method for occluding an aneurysm comprising: (a) inserting a first longitudinal section of a flexible longitudinal embolic member into an aneurysm and rotating this first longitudinal section around its longitudinal axis; and (b) inserting a second longitudinal section of a flexible longitudinal embolic member into the aneurysm; wherein this second longitudinal section comprises a series of linked embolic members; wherein the second longitudinal section is distally connected to the first longitudinal section; and wherein the combination of inserting the second section into the aneurysm and rotating the first section causes the first and second sections to create a densely-packed embolic mass within the aneurysm.

We now discuss the specific components of FIGS. 19 through 21 in detail. FIG. 19 shows an example of an occlusive device that comprises: a first longitudinal lumen (e.g. catheter) 1904 that is configured to be inserted into a blood vessel that is the parent vessel from which an aneurysm has formed; a second longitudinal lumen 1901 that is configured to be inserted into this blood vessel; a flexible loop 1903 that is configured to travel through the first and/or second longitudinal lumens and be inserted into the aneurysm sac, wherein a first strand (or side) of loop 1903 is rotated around its longitudinal axis while a second strand (or side) of loop 1903 is inserted into the aneurysm sac; and a series of embolic members (including 1902) which are configured to travel through longitudinal lumen 1901 and be inserted into the aneurysm sac, wherein these embolic members are connected by the second strand (or side) of flexible loop 1903.

As shown in FIG. 20, rotation of a first strand (or side) of flexible loop 1903 combined with insertion of a second strand (or side) of loop 1903 with attached embolic members (such as 1902) into the aneurysm sac creates an entwined embolic mass within the aneurysm sac. In this example, a first strand (or side) of flexible loop 1903 travels through first longitudinal lumen (e.g. catheter) 1904 and a second strand (or side) of flexible loop 1903 travels through second longitudinal lumen 1901. In another example, an occlusive device may have only one longitudinal lumen and both strands (or sides) of a flexible loop can travel through the same longitudinal lumen.

In an example, the rate at which the first strand of loop 1903 is rotated, the rate at which one or both strands of loop 1903 are inserted into an aneurysm sac, or both rates can be selected based on the size and shape of the aneurysm sac. In an example, one or both of these rates can be varied during implantation. In an example, the rate at which the first strand of loop 1903 is rotated can vary from a faster rate to a slower rate during implantation. In an example, the rate at which the second strand of loop 1903 is pushed into the aneurysm sac can vary from a slower rate to a faster rate during implantation.

In an example, embolic members (including 1902) can be evenly-spaced along the second strand (or side) of flexible loop 1903. In an example, the spacing of embolic members (including 1902) along the second strand (or side) of flexible loop 1903 can be selected based on the size and shape of the aneurysm to be occluded. In an example, the spacing of embolic members (including 1902) along the second strand (or side) of flexible loop 1903 can vary from the distal end of flexible loop 1903 to the proximal end of flexible loop 1903.

As shown in FIG. 21, a distal portion of flexible loop 1903 can be detached and left in the aneurysm sac when a sufficient volume of embolic members has accumulated in the entwined mass. In an example, embolic members (including 1902) can adhere to each other so that the entwined mass that they form is more cohesive and stable. This can reduce the probability of portions of the loop or embolic members prolapsing into the parent blood vessel. Relevant variations discussed elsewhere in this disclosure or in priority-linked disclosures can also be applied to this example.

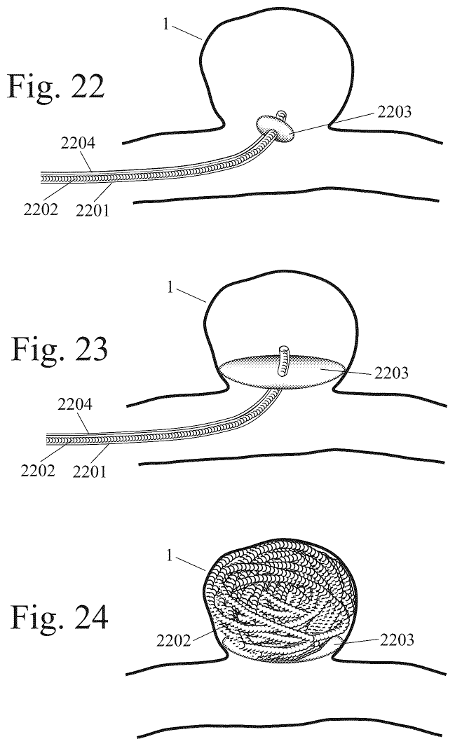

FIGS. 22 through 24 show an example of a device and method to occlude an aneurysm which can be described as aneurysm coil jailing using an expandable torus. More specifically, FIGS. 22 through 24 show an example of a device to occlude an aneurysm comprising: (a) a longitudinal flexible embolic member that is configured to be inserted into an aneurysm, wherein accumulation of this embolic member within the aneurysm occludes the aneurysm; (b) an embolic-member-delivering lumen (e.g. catheter) which is configured to be inserted into a blood vessel, wherein the longitudinal flexible embolic member travels through this lumen (e.g. catheter) in order to be inserted into the aneurysm; and wherein this embolic-member-delivering lumen (e.g. catheter) is withdrawn from the blood vessel after the longitudinal flexible member has accumulated within the aneurysm; (c) a toroidal inflatable member that is configured to be inserted into the aneurysm; wherein this inflatable member substantially occludes the neck of an aneurysm when the inflatable member is inflated after being inserted into the aneurysm; and wherein the longitudinal flexible embolic member is inserted into the aneurysm through the central opening of the toroidal inflatable member after the toroidal member has been inflated; and (d) a flowable-substance-delivering lumen (e.g. catheter) which is configured to be inserted into a blood vessel, wherein a flowable substance travels through this lumen (e.g. catheter) into the toroidal inflatable member in order to inflate the toroidal member after it has been inserted into the aneurysm; and wherein this flowable-substance-delivering lumen (e.g. catheter) is withdrawn from the blood vessel after the toroidal inflatable member has been inflated within the aneurysm.

FIGS. 22 through 24 also show an example of a device to occlude an aneurysm comprising: (a) a longitudinal lumen (e.g. catheter) that is configured to be inserted into a blood vessel, wherein this blood vessel is the parent vessel from which an aneurysm has formed; (b) a longitudinal flexible embolic member that is configured to travel through the longitudinal lumen (e.g. catheter) and be inserted into the aneurysm sac; and (c) an expandable toroidal member that is configured to travel through the longitudinal lumen (e.g. catheter), be inserted into the aneurysm sac, and to be expanded within the aneurysm sac; wherein this toroidal member is configured to substantially occlude the aneurysm neck after it is expanded; wherein the longitudinal flexible embolic member is inserted into the aneurysm sac through the central opening of the expandable toroidal member; and the expandable toroidal member prevents the longitudinal flexible member from protruding into the parent vessel of the aneurysm.

In an example, the longitudinal lumen (e.g. catheter) can be a removable catheter. In an example, the longitudinal flexible embolic member can be a coil. In an example, the expandable toroidal member can be expanded by inflation with a gas. In an example, the expandable toroidal member can be expanded by filling it with a liquid or gel. In an example, the expandable toroidal member can self expand because it comprises shape memory material. In an example, the expandable toroidal member can remain in the aneurysm sac after implantation of the device. In an example, the expandable toroidal member can be expanded by filling it with a liquid or gel which solidifies after filling and wherein this solidification further helps to keep the expandable toroidal member in the aneurysm sac. In an example, the longitudinal flexible expandable member can adhere to the expandable toroidal member when the two members come into contact with each other and this adhesion can further help to keep the expandable toroidal member within the aneurysm sac.

We now discuss the specific components of the example shown in FIGS. 22 through 24. FIG. 22 shows an occlusive device which comprises: a first longitudinal lumen (e.g. catheter) 2201 that is configured to be inserted into a blood vessel from which an aneurysm sac 1 has formed; a second longitudinal lumen (e.g. catheter) 2204 that is configured to be inserted into this blood vessel; a longitudinal flexible embolic member 2202 that is configured to travel through the first longitudinal lumen (e.g. catheter) 2201 and be inserted into the aneurysm sac; and an expandable toroidal member 2203 that is configured to be inserted into and expanded within aneurysm sac 1.

As shown in FIG. 23, toroidal member 2203 can be expanded by being filled with a gas, liquid, or gel that is delivered via second longitudinal lumen (e.g. catheter) 2204. In an example, toroidal member 2203 can be configured to substantially occlude the aneurysm neck after it is expanded. In an example toroidal member 2203 can be expanded in a radial-expansion plane that is substantively parallel with the plane defined by the central circumference of the aneurysm neck. In an example, toroidal member 2203 can be expanded at a distance from the central circumference of the aneurysm neck from which its radial expansion contacts the aneurysm sac at the sac's maximum circumference. In an example, toroidal member 2203 can be expanded at a distance from the central circumference of the aneurysm neck from which its radial expansion contacts the aneurysm sac at a sac circumference that is greater than the central circumference of the aneurysm neck.

In an example, longitudinal flexible embolic member 2202 can be inserted into aneurysm sac 1 through a central opening in toroidal member 2203. In an example, longitudinal lumen (e.g. catheter) 2201 can fit snugly through this opening so that longitudinal flexible embolic member 2202 does not prolapse out from this opening into the parent vessel. In an example, toroidal member 2203 can prevent longitudinal flexible member 2202 from prolapsing into the parent blood vessel of the aneurysm. In an example, a plurality of embolic members can be inserted into the aneurysm sac through this central opening and toroidal member 2203 can have a sufficiently tight fit with the aneurysm walls that none of the plurality of embolic members escape into the parent blood vessel.

In the example that is shown in FIGS. 22 through 24, longitudinal lumen (e.g. catheter) s 2201 and 2204 are parallel catheters which are removed from the body after the aneurysm is occluded. In this example, longitudinal flexible embolic member 2202 is an embolic coil. In this example, toroidal member 2203 is expanded by being filled with a gas, liquid, or gel. In an example, a toroidal member can self expand because it comprises shape memory material, thereby eliminating the need for longitudinal lumen (e.g. catheter) 2204. As shown in FIG. 24, expandable toroidal member 2203 can remain in the aneurysm sac after implantation, but longitudinal lumen (e.g. catheter) s 2201 and 2204 can be removed. In an example, longitudinal flexible expandable member 2202 can adhere to toroidal member 2203 to help keep toroidal member from protruding into the blood vessel. Relevant variations discussed elsewhere in this disclosure or in priority-linked disclosures can also be applied to this example.

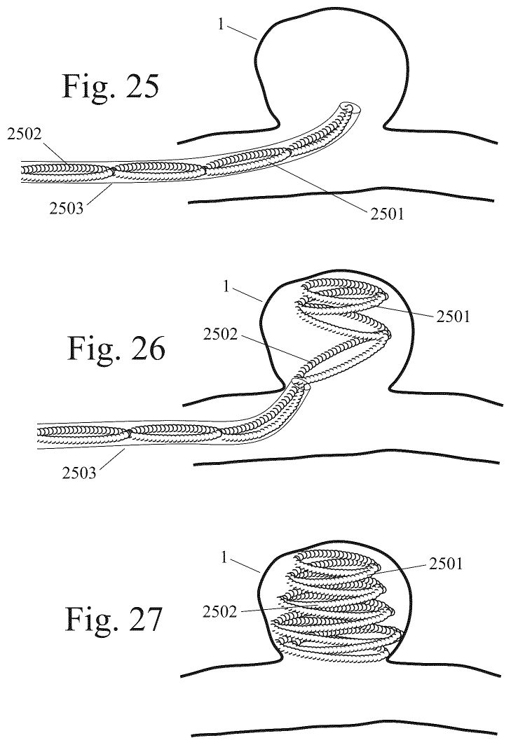

FIGS. 25 through 27 show an example of a device and method to occlude an aneurysm which can be described as aneurysm occlusion using loops which are substantially-parallel to the aneurysm neck. More specifically, FIGS. 25 through 27 show an example of a device to occlude an aneurysm comprising: (a) a longitudinal lumen (e.g. catheter) that is configured to be inserted into a blood vessel; and (b) a flexible longitudinal embolic member that is configured to travel through the lumen in order to be inserted through the lumen into an aneurysm; wherein this flexible longitudinal embolic member comprises a longitudinal series of loops; wherein these loops are laterally compressed as they travel through the lumen and laterally expand within the aneurysm after they exit the lumen; wherein adjacent loops along the longitudinal series are connected to each other by junctions; and wherein loop sizes are selected to be substantially equal to the circumferences of the aneurysm sack at the expected implant locations so that junction locations alternate from one side of the aneurysm to the other side of the aneurysm as successive loops accumulate within the aneurysm.

FIGS. 25 through 27 also show an example of a device to occlude an aneurysm comprising: (a) a longitudinal lumen (e.g. catheter) that is configured to be inserted into a blood vessel; and (b) a flexible longitudinal embolic member that is configured to travel through the lumen in order to be inserted through the lumen into an aneurysm; wherein this flexible longitudinal embolic member comprises a longitudinal series of loops; wherein these loops are laterally compressed as they travel through the lumen and laterally expand within the aneurysm after they exit the lumen; and wherein loop sizes are selected to be substantially equal to the circumferences of the aneurysm sack at the expected implant locations so that the planes formed by loops are substantially within plus or minus 30 degrees of being parallel to the plane formed by the aneurysm neck.

FIGS. 25 through 27 also show an example of a device to occlude an aneurysm comprising: (a) a longitudinal lumen (e.g. catheter) that is configured to be inserted into a blood vessel, wherein this blood vessel is the parent vessel from which an aneurysm has formed; (b) a first longitudinal flexible embolic member that is configured to travel through the longitudinal lumen and be inserted into the aneurysm sac; (c) a second longitudinal flexible embolic member that is configured to travel through the longitudinal lumen and be inserted into the aneurysm sac; wherein the first and second embolic members are contiguous and/or connected to each other at their distal ends; wherein the first and second embolic members are connected at a plurality of locations along the lengths of these embolic members; wherein segments of the first and second embolic members that are not connected move away from each other after they exit the longitudinal lumen, thereby forming loops within the aneurysm sac; and wherein a plane spanned by a loop is substantively parallel to the plane spanned by the central circumference of the aneurysm neck.

In an example, the longitudinal lumen (e.g. catheter) can be a removable catheter. In an example, the embolic members can be coils. In an example, a plane spanned by a loop can be within 30 degrees of being parallel to the plane spanned by the central circumference of the aneurysm neck. In an example, a plane spanned by a loop can be within 45 degrees of being parallel to the plane spanned by the central circumference of the aneurysm neck. In an example, the orientations of sequential loops formed within the aneurysm sac can sequentially alternate from one side of the aneurysm sac to the other side of the aneurysm sac. In an example, the accumulating loops can form a beehive-shaped mass of loops within the aneurysm sac and not prolapse out from the aneurysm neck into the parent vessel.

We now discuss the specific components of the example that is shown in FIGS. 25 through 27. FIG. 25 shows an occlusive device comprising: a longitudinal lumen (e.g. catheter) 2503 that is configured to be inserted into a blood vessel, wherein this blood vessel is the parent vessel from which an aneurysm sac 1 has formed; a first longitudinal flexible embolic member 2501 that is configured to travel through the longitudinal lumen and be inserted into aneurysm sac 1; and a second longitudinal flexible embolic member 2502 that is configured to travel through the longitudinal lumen and be inserted into aneurysm sac 1. In this example, embolic members 2501 and 2502 are contiguous to each other at their distal ends. In this example, embolic members 2501 and 2502 are connected at a plurality of locations along their lengths. As shown in FIG. 26, segments of embolic members 2501 and 2502 that are not connected to each other move away from each other after they exit the longitudinal lumen, thereby forming connected loops within aneurysm sac 1.

In this example, a plane that is spanned by one of these loops can become (in its final configuration) substantively parallel to the plane that is spanned by the central circumference of the aneurysm neck. In an example, this final configuration can occur after multiple such loops have been formed and accumulated in the aneurysm sac. In an example, a plane spanned by a loop can become within plus-or-minus 30 degrees of being parallel to the plane spanned by the central circumference of the aneurysm neck. In an example, virtual extension of the plane spanned by a loop can form an acute angle with the virtual extension of the plane spanned by the central circumference of the aneurysm neck. In an example, this angle can be less than 30 degrees. In an example, a plane spanned by a loop can become within plus-or-minus 45 degrees of being parallel to the plane spanned by the central circumference of the aneurysm neck. In an example, virtual extension of the plane spanned by a loop can form an acute angle with the virtual extension of the plane spanned by the central circumference of the aneurysm neck. In an example, this angle can be less than 45 degrees.

As shown in FIG. 27, the accumulation of sequential loops with sides comprised of embolic members 2501 and 2502 can form an embolic mass with a zigzag pattern within the aneurysm sac. In this example, the acute angles formed between the zigzagging loops are less than 30 degrees. In an example, the orientations of sequential loops formed within aneurysm sac 1 can sequentially alternate from one side of aneurysm sac 1 to the other side of aneurysm sac 1. As shown in FIG. 27, accumulating loops can form a “beehive-shaped” mass of loops within the aneurysm sac. In an example, the probability of having embolic members prolapse into the parent blood vessel can be reduced (as compared to free-form spiraling coils in the prior art) by a combination of: (a) creating loops which frictionally engage the aneurysm sac walls at one or more locations; (b) creating loops with an orientation which is substantially parallel to the plane defined by the central circumference of the aneurysm neck; and (c) creating loops which are contiguous and interconnected. Relevant variations discussed elsewhere in this disclosure or in priority-linked disclosures can also be applied to this example.

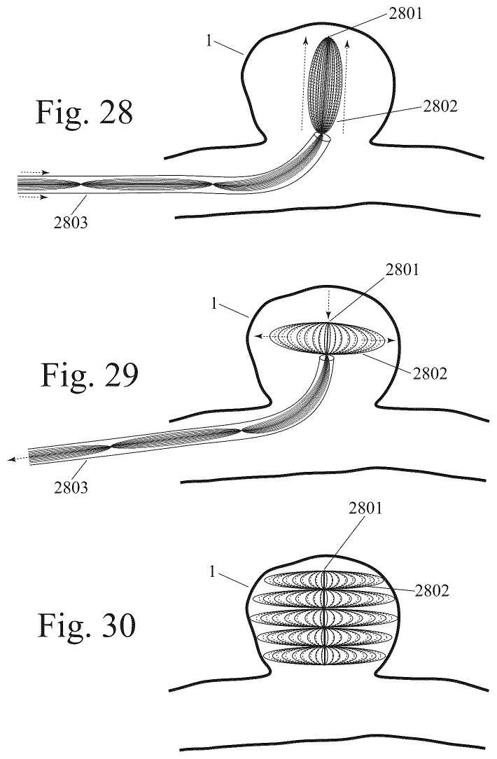

FIGS. 28 through 30 show an example of a device and method to occlude an aneurysm which can be described as aneurysm occlusion using multiple centrally-aligned ellipsoids. More specifically, FIGS. 28 through 30 show an example of a device to occlude an aneurysm comprising: (a) a longitudinal lumen (e.g. catheter) that is configured to be inserted into a blood vessel, wherein this lumen has a longitudinal axis spanning from its proximal end to its distal end and wherein the distal end is first inserted into the blood vessel; and (b) a plurality of longitudinally-linked configuration-changing embolic members which are configured to travel through the longitudinal lumen and to be inserted into an aneurysm; wherein each shape-changing embolic member has its own internally-referenced Z axis, X axis, and Y axis; wherein its Z axis is substantially parallel to the longitudinal axis of the longitudinal lumen as the embolic member travels through the longitudinal lumen, its X axis is substantially perpendicular to its Z axis, and its Y axis is substantially perpendicular to both its Z axis and X axis; wherein each configuration-changing embolic member has a first configuration as the member travels through the longitudinal lumen and a second configuration within the aneurysm after it exits the longitudinal lumen; wherein the distance of the embolic member spanning its Z axis is greater than the distance of the embolic member spanning its X axis or Y axis in the first configuration; wherein the distance of the embolic member spanning its Z axis is less than the distance of the embolic member spanning its X axis or Y axis in the second configuration; wherein the cross-sectional shape of the embolic member in an X-Z plane is substantially elliptical, oval, or another arcuate non-circular shape in the first configuration, with the longer dimension of the ellipse, oval, or another arcuate non-circular shape being along its Z axis; and wherein the cross-sectional shape of the embolic member in the X-Z plane is substantially elliptical, oval, or another arcuate non-circulate shape in the second configuration, with the longer dimension of the ellipse, oval, or another arcuate non-circular shape being along its X axis.

FIGS. 28 through 30 also show an example of a device to occlude an aneurysm comprising: (a) a longitudinal lumen (e.g. catheter) that is configured to be inserted into a blood vessel, wherein this blood vessel is the parent vessel from which an aneurysm has formed; and (b) a series of connected embolic ellipsoids, wherein these embolic ellipsoids are configured to travel in series through the longitudinal lumen and to be inserted into the aneurysm sac; wherein an embolic ellipsoid has a first orientation as it travels through the longitudinal lumen; wherein an embolic ellipsoid has a second orientation after it exits the longitudinal lumen; wherein in the first orientation the longitudinal axis of the ellipsoid is substantively parallel to the longitudinal axis of the longitudinal lumen; wherein in the second orientation the longitudinal axis of the ellipsoid is substantially perpendicular to its prior orientation traveling through the longitudinal lumen.

In an example, the longitudinal lumen (e.g. catheter) can be a removable catheter. In an example, the embolic ellipsoid can be a wire structure. In an example, the embolic ellipsoid can have a first orientation when it exits the aneurysm sac but then be compressed into a second orientation. In an example, the series of connected embolic ellipsoids can form a stack of connected ellipsoids which share a common central axis within the aneurysm sac. In an example, the series of connected embolic ellipsoids can form a stack of connected ellipsoid disks which share a common central axis within the aneurysm sac and fill a greater volume of the aneurysm sac than would be filled by a single hollow mesh structure with a similar size perimeter as the stack of connected ellipsoid disks. In an example, at least one of the connected ellipsoid disks can have a circumference that is larger than the circumference of the aneurysm neck in order to help keep the structure within the aneurysm sac.

We now discuss the components of the example that is shown in FIGS. 28 through 30 in detail. FIG. 28 shows an occluding device that comprises: a longitudinal lumen (e.g. catheter) 2803 that is configured to be inserted into a blood vessel, wherein this blood vessel is the parent vessel from which an aneurysm has formed; and a series of connected embolic ellipsoids (including 2802). In this example, the embolic ellipsoids (including 2802) are configured to travel in series through longitudinal lumen 2803 and be inserted into the aneurysm sac.

As shown in FIG. 29, each embolic ellipsoid (including 2802) can have a first orientation as it travels through lumen (e.g. catheter) 2803 and a second orientation after it exits lumen 2803 inside aneurysm sac 1. In an example, each embolic ellipsoid can have a longitudinal axis such as 2801 for embolic ellipsoid 2802. In an example, in the first orientation, the longitudinal axis (such as 2801) of the ellipsoid (such as 2802) can be substantively-parallel to the longitudinal axis of lumen 2803. In an example, in the second orientation, the longitudinal axis (such as 2801) of the ellipsoid (such as 2802) can be substantially-perpendicular to its prior orientation traveling through lumen 2803.

In an example, an embolic ellipsoid (such as 2802) can be oriented as it travels through lumen (e.g. catheter) 2803 such that its longest axis is substantially-parallel to the longitudinal axis of lumen 2803. In an example, an embolic ellipsoid (such as 2802) can be compressed and/or reoriented after it exits lumen 2803 so that its longest axis becomes substantially-parallel to the plane that is defined by the central circumference of the aneurysm neck. In an example, the longitudinal axes (such as 2801) of the embolic ellipsoids (such as 2802) as these ellipsoids travel through lumen 2803 can become the virtual lateral axes (still 2801) of these embolic ellipsoids (such as 2802) when these ellipsoids are compressed and/or reoriented after they exit lumen 2803.

In example, the longitudinal axes (including 2802) of these ellipsoids (including 2802) can be compressed after the ellipsoids exit lumen (e.g. catheter) 2803. In an example, this compression can be caused by movement of a wire, fiber, or other longitudinal flexible member that is connected to the ellipsoids. In an example, this compression can be caused by contact between the aneurysm wall and the ellipsoids. In an example, the embolic ellipsoids (including 2802) can have a shape memory and a prior shape to which they return after their release from lumen 2803. In an example, their return to a prior shape can cause the change in their orientation and/or compression after they exit lumen 2803. In this example, the embolic ellipsoids (including 2802) are wire structures.

As shown in FIG. 30, a series of connected embolic ellipsoids (including 2802) can form a stack of connected ellipsoids which share a common central axis within aneurysm sac 1. In an example, a series of connected embolic ellipsoids can form a stack of connected ellipsoid disks which share a common central axis within the aneurysm sac. In an example, this stack of connected ellipsoids can fill a greater volume of the aneurysm sac than would be filled by a single hollow-mesh structure (such as a wire-mesh single sphere or ellipsoid that is expanded with an aneurysm sac) with a similar-size perimeter as the combined stack of connected ellipsoid disks. As shown in FIG. 30, at least one of the connected ellipsoids has a circumference that is larger than the circumference of the aneurysm neck in order to help keep the stack within the aneurysm sac. Relevant variations discussed elsewhere in this disclosure or in priority-linked disclosures can also be applied to this example.

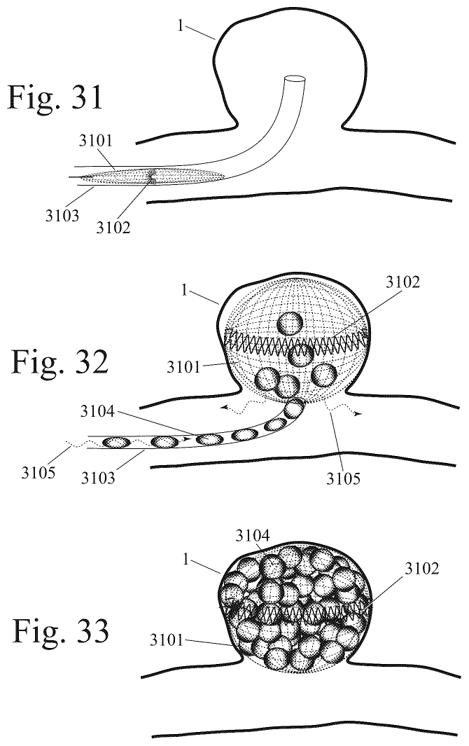

FIGS. 31 through 33 show an example of a device and method to occlude an aneurysm which can be described as a “Saturn-shaped” device for aneurysm occlusion. More specifically, FIGS. 31 through 33 show an example of a device to occlude an aneurysm comprising: (a) a longitudinal lumen (e.g. catheter) that is configured to be inserted into a blood vessel; (b) a flexible expandable member that is configured to travel through the longitudinal lumen, be inserted into an aneurysm, and then be expanded within the aneurysm sack; wherein this flexible expandable member is selected from the group consisting of a net, a mesh, a lattice, and a balloon with holes; wherein this flexible expandable member is sufficiently flexible to substantively conform to the contours of the walls of the aneurysm sack after the flexible expandable member is expanded within the aneurysm; and wherein this flexible expandable member is permeable to liquid; (c) a resilient expandable member that is configured to travel through the longitudinal lumen, be inserted into the aneurysm, and then be expanded within the aneurysm sack; wherein this resilient expandable member resists contraction after it has been expanded; wherein a plane formed by the expanding circumference of this resilient expandable member is substantially parallel to the plane that centrally spans the circumference of the aneurysm neck; wherein a plane formed by the expanding circumference of this resilient expandable member spans the aneurysm sack at the sack's largest circumference parallel to the plane that centrally spans the circumference of the aneurysm neck; and wherein expansion of the resilient expandable member resiliently holds a central portion of the flexible expandable member against the walls of the aneurysm so that the flexible expandable member does not slip out of the aneurysm sack; and (d) a plurality of individual embolic members that are configured to travel through the longitudinal lumen, be inserted into the flexible expandable member within the aneurysm, and accumulate within the flexible expandable member; wherein the flexible expandable member does not allow the embolic members to escape out from the flexible expandable member; and wherein accumulation of the plurality of embolic members inside the flexible expandable member causes the flexible expandable member to expand.

FIGS. 31 through 33 also show an example of a device to occlude an aneurysm comprising: (a) a longitudinal lumen (e.g. catheter) that is configured to be inserted into a blood vessel, wherein this blood vessel is the parent vessel from which an aneurysm has formed; (b) an expandable flexible net or mesh, wherein this expandable flexible net or mesh is configured to travel through the longitudinal lumen and to be inserted into the aneurysm sac, and wherein this net or mesh is sufficiently flexible to substantially conform to the walls of an irregularly shaped aneurysm sac after the net or mesh has been expanded; (c) a plurality of embolic members, wherein these embolic members are configured to travel through the longitudinal lumen and to be inserted into the net or mesh within the aneurysm sac; wherein these embolic members do not escape from the net or mesh; and wherein the net or mesh is expanded by the accumulation of embolic members inside the net or mesh; and (d) an expandable resilient structure, wherein this expandable resilient structure is configured to travel through the longitudinal lumen and to be inserted into the aneurysm sac; wherein this structure comes into engaging contact with the central circumference of the aneurysm sac when this structure is expanded; wherein this structure resists compression after it has been expanded; and wherein expansion of this structure also engages the net or mesh so as to prevent the net or mesh from slipping out from the aneurysm sac.

In an example, the longitudinal lumen (e.g. catheter) can be a removable catheter. In an example, the net or mesh can be a wire net or mesh. In an example, the net or mesh can be a polymer net or mesh. In an example, the expandable resilient structure can be a stent. In an example, the expandable resilient structure can be attached to the net or mesh. In an example, the expandable resilient structure can be inside the net or mesh. In an example, the plurality of embolic members can be conveyed through the longitudinal lumen by means of a liquid flow and the net or mesh can be sufficiently porous so as to let the liquid escape through the net or mesh but does not let the embolic members escape through the net or mesh. In an example, the total volume of an aneurysm sac can be X cubic units, wherein Y cubic units of the volume of the aneurysm would be filled by the largest-volume sphere that can be fitted into the aneurysm without stretching the aneurysm walls, wherein Z cubic units of the volume of the aneurysm can be filled by the net or mesh; and wherein Z>[Y+0.5 (X−Y)]. In an example, the total volume of an aneurysm sac can be X cubic units, wherein Y cubic units of the volume of the aneurysm would be filled by the largest-volume ellipsoid that can be fitted into the aneurysm without stretching the aneurysm walls, wherein Z cubic units of the volume of the aneurysm can be filled by the net or mesh; and wherein Z>[Y+0.5(X−Y)].

We now discuss the specific components of the example that is shown FIGS. 31 through 33. FIGS. 31 through 33 show an occlusive device comprising: longitudinal lumen (e.g. catheter) 3103 that is inserted into a blood vessel from which an aneurysm sac 1 has formed; expandable flexible net or mesh 3101 that travels through lumen 3103 into aneurysm sac 1, wherein net or mesh 3101 is sufficiently flexible to substantially conform to the walls of aneurysm sac 1; a plurality of embolic members (including 3104) which travel through lumen 3103 into net or mesh 3101, wherein these embolic members (including 3104) do not escape from net or mesh 3101 and wherein net or mesh 3101 is expanded by the accumulation of embolic members (including 3104) inside net or mesh 3101; and expandable resilient structure 3102 which travels through lumen 3103 and is expanded within aneurysm sac 1.

In an example, a flexible expandable member can be selected from the group consisting of a net, a mesh, and a lattice. In an example, a flexible expandable member can be selected from the group consisting of a balloon, a bag, and a liner. In an example, a flexible expandable member is made of a polymer, a metal, or a combination thereof.

As shown in FIG. 32, structure 3102 comes into engaging contact with the central circumference of aneurysm sac 1 when structure 3102 is expanded. Structure 3102 can be expanded sufficiently to frictionally engage the walls of aneurysm sac 1, but not expanded so much that it risks puncturing the walls of aneurysm sac 1. In an example, structure 3102 can have a rounded perimeter. In an example, structure 3102 can have a bioadhesive coating which adheres to the aneurysm walls to further engage them. Structure 3102 also resists compression after it expands. In this example, expansion of structure 3102 also engages net or mesh 3101 to prevent net or mesh 3101 from slipping out from aneurysm sac 1.

In this example, longitudinal lumen (e.g. catheter) 3103 is a removable catheter. In this example, net or mesh 3101 is a wire net or mesh. In an example, net or mesh 3101 can be a polymer net or mesh. In this example, expandable resilient structure 3102 is integrated with net or mesh 3101. In an example, the plurality of embolic members (including 3104) can be conveyed through lumen 3103 by means of a liquid flow. In an example, net or mesh 3101 can be sufficiently porous so as to let the liquid escape through net or mesh 3101 but not so porous that it lets embolic members (including 3104) escape through net or mesh 3101. In an example, embolic members (including 3104) can be compressed as they travel through lumen 3103 but these embolic members (including 3104) can expand when released from lumen 3103. This can help to prevent embolic members from escaping out of net or mesh 3101.

In an example, this device can also include a closure mechanism which is integrated into net or mesh 3101 to further prevent embolic members (including 3104) from escaping from net or mesh 3101 through the opening by which they were inserted into net or mesh 3101. In an example, this closure mechanism can comprise a one-way valve that automatically lets embolic members into the net or mesh but does not let them out. In an example, this closure mechanism can require action by a user during the procedure to close off the opening. In an example, this closure mechanism can comprise a drawstring, loop, seal, fusible member, adhesive, snap, clip, valve, or cap.

As shown in FIGS. 31 through 33, an aneurysm sac can be irregular in shape. An aneurysm sac with an irregular shape will not be completely filled or spanned by a spherical or ellipsoid mass without stretching the aneurysm walls. In an example, the total volume of an aneurysm sac can be X cubic units (e.g. cubic millimeters). In an example, the maximum volume of the aneurysm which can be filled or spanned by a spherical or ellipsoid mass without stretching the aneurysm walls is Y cubic units (e.g. cubic millimeters). In an example, the device shown in FIGS. 31 through 33 can fill more of the aneurysm than a spherical or ellipsoid mass because the net or mesh is sufficiently flexible to fill or span the irregular perimeter of the aneurysm sac. This can have clinical benefits, such as reducing the chances of recanalization within the aneurysm sac. In an example, this device can fill or span more than 50% of the aneurysm volume which remains unfilled by a sphere or ellipsoid. In an example, this device can fill or span Z cubic units (e.g. cubic millimeters) of the volume of the aneurysm, wherein Z>[Y+0.5 (X−Y)].

In an example, net or mesh 3101 can be compressed as it travels through lumen (e.g. catheter) 3103 and then be expanded within aneurysm sac 1 after it is released from lumen 3103. In an example, net or mesh 3101 can be folded as it travels through lumen 3103 and then be unfolded within aneurysm sac 1. In an example, net or mesh 3101 can be relatively loose or relaxed (in a lower-energy state) as it travels through lumen 3103 and then be stretched or tense (in a higher-energy state) within aneurysm sac 1. In an example, net or mesh 3101 can be elastic or stretchable. In an example, net or mesh 3101 can be sufficiently elastic or stretchable that it expands when filled with an accumulation of embolic members (including 3104), but not so elastic or stretchable that it allows embolic members (including 3104) to escape. In an example, net or mesh 3101 can be a balloon with holes, wherein the holes are of sufficient size to let liquid escape, but not so large that they let embolic members escape.

In an example, expandable resilient structure 3102 can be a ring-like expandable stent. In an example, expandable resilient structure 3102 can be a cylindrical expandable stent. In an example, expandable resilient structure can be an ellipsoid expandable stent. In an example, expandable resilient structure 3102 can be a wire mesh stent. In an example, expandable resilient structure 3102 can be centrally-located so as to expand from the center of net or mesh 3101. In an example, expandable resilient structure 3102 can be inside net or mesh 3101 and thereby hold net or mesh against the aneurysm wall when structure 3102 is expanded. In an example, expandable resilient structure 3102 can be attached to net or mesh 3101 and thereby hold net or mesh 3101 within the aneurysm sac when structure 3102 is expanded. In an example the expandable resilient structure 3102 can be radially-expanded in plane which is substantially parallel to the plane that is defined by the central circumference of the aneurysm neck. In an example, expandable resilient structure 3102 can be expanded by a removable balloon. In an example, expandable resilient structure 3102 can self-expand when released from lumen (e.g. catheter) 3103.

In an example, embolic members (including 3104) can be a plurality of soft, compressible members such as microsponges or blobs of gel. In an example, embolic members (including 3104) can be a plurality of hard, uncompressible members such as hard polymer spheres or beads. In an example, embolic members (including 3104) can be conveyed through lumen (e.g. catheter) 3103 in a fluid flow, wherein the fluid escapes out from net or mesh 3101 and the embolic members are retained within net or mesh 3101. In an example, embolic members (including 3104) can be conveyed through lumen 3103 by means of a moving belt or wire loop. In an example, embolic members (including 3104) can be conveyed through lumen 3103 by means of an Archimedes screw.

In an example, the combination of (a) a flexible, non-resilient net or mesh 3101 that spans substantially the entire perimeter of the aneurysm sac 1 and (b) a resilient expandable structure 3102 that only spans a central portion of the circumference of the aneurysm sac 1 can create a device that is sufficiently flexible to substantially fill the entire volume of an irregularly-shaped aneurysm sac, but also sufficiently resilient so as to compress against the aneurysm walls and not slip out of the aneurysm sac. Relevant variations discussed elsewhere in this disclosure or in priority-linked disclosures can also be applied to this example.

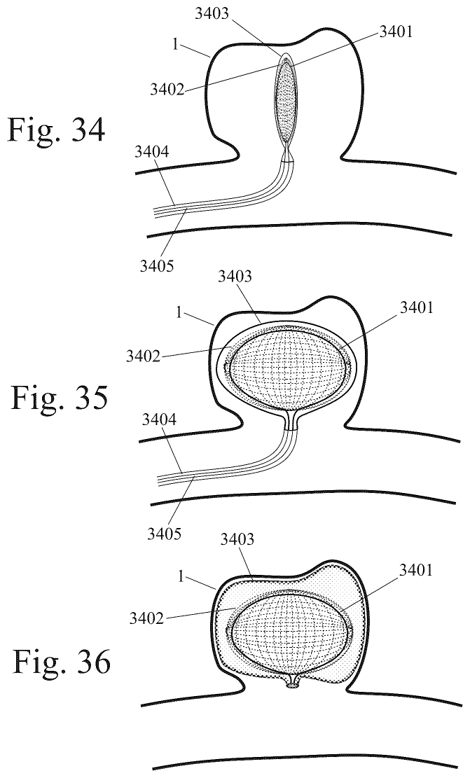

FIGS. 34 through 36 show an example of a device and method to occlude an aneurysm which can be described as using concentric resilient and non-resilient intrasaccular members for aneurysm occlusion. More specifically, FIGS. 34 through 36 show an example of a device to occlude an aneurysm comprising: (a) a longitudinal lumen (e.g. catheter) that is configured to be inserted into a blood vessel; (b) a resilient expandable member that is configured to travel through the longitudinal lumen, be inserted into an aneurysm sack, and then be expanded within the aneurysm sack; wherein this resilient expandable member resists contraction after it has been expanded; and wherein this resilient expandable member has a post-expansion shape that is selected from the group consisting of spherical, ellipsoidal, toroidal, compressed-sphere shaped, egg shaped, Saturn shaped, hour-glass shaped, peanut shaped, beehive shaped and geodesic; and (c) a flexible expandable member that is configured to travel through the longitudinal lumen, be inserted into the aneurysm sack, and then be expanded within the aneurysm sack; wherein the resilient expandable member is inside the flexible expandable member; wherein the resilient expandable member is expanded before or while the flexible expandable member is expanded; and wherein the flexible expandable member is sufficiently flexible to substantively conform to the contours of the walls of the aneurysm sack when the flexible expandable member is expanded within the aneurysm.

FIGS. 34 through 36 also show an example of a device to occlude an aneurysm comprising: (a) a longitudinal lumen (e.g. catheter) that is configured to be inserted into a blood vessel, wherein this blood vessel is the parent vessel from which an aneurysm has formed; (b) an expandable flexible member, wherein this expandable flexible member is configured to travel through the longitudinal lumen and to be inserted into the aneurysm sac; and wherein this flexible member is sufficiently flexible to substantially conform to the walls of an irregularly-shaped aneurysm sac after the flexible member has been expanded; and (c) an expandable resilient structure, wherein this expandable resilient structure is configured to travel through the longitudinal lumen and to be inserted into the aneurysm sac; wherein this structure is expanded inside the expandable flexible member; and wherein this structure resists compression after it has been expanded.

In an example, the longitudinal lumen (e.g. catheter) can be a removable catheter. In an example, the expandable flexible member can be a balloon. In an example, the expandable flexible member can be a net or mesh. In an example, the expandable flexible member can be expanded by being filled with a liquid, gel, gas, or embolic members. In an example, the expandable resilient structure can be a stent. In an example, the expandable resilient structure can be substantially spherical or elliptical. In an example, the expandable resilient structure can be expanded before the expandable flexible member is expanded. In an example, the expandable resilient structure and the expandable flexible member can be expanded at substantially the same time. In an example, the total volume of an aneurysm sac can be X cubic units, wherein Y cubic units of the volume of the aneurysm would be filled by the largest-volume sphere that can be fitted into the aneurysm without stretching the aneurysm walls, wherein Z cubic units of the volume of the aneurysm can be filled by the expandable and flexible member; and wherein Z>[Y+0.5 (X−Y)]. In an example, the total volume of an aneurysm sac can be X cubic units, wherein Y cubic units of the volume of the aneurysm would be filled by the largest-volume ellipsoid that can be fitted into the aneurysm without stretching the aneurysm walls, wherein Z cubic units of the volume of the aneurysm can be filled by the expandable flexible member; and wherein Z> [Y+0.5 (X−Y)].

We now discuss the specific components of the example shown in FIGS. 34 through 36. FIGS. 34 through 36 show an example of an occlusive device comprising: an outer longitudinal lumen (e.g. catheter) 3404 that is configured to be inserted into a blood vessel from which aneurysm sac 1 has formed; an inner longitudinal lumen 3405 within longitudinal lumen 3404; an expandable flexible member 3403 that is inserted and expanded within aneurysm sac 1, wherein flexible member 3403 is sufficiently flexible to substantially conform to the walls of irregularly-shaped aneurysm sac 1; and an expandable resilient structure 3402 that is expanded within flexible member 3403 and resists compression after expansion.

In an example, expandable resilient structure 3402 can be spherical or elliptical. In an example, expandable resilient structure 3402 can be an expandable wire mesh or stent. In an example, expandable resilient structure 3402 can be radially-expanded in plane which is substantially parallel to the plane that is defined by the central circumference of the aneurysm neck. In an example, resilient structure 3402 can be expanded by inflation of a balloon 3401 inside resilient structure 3402. In an example, balloon 3401 can be inflated by a fluid or gas that is delivered via lumen (e.g. catheter) 3404 or lumen (e.g. catheter) 3405. In an example, resilient structure 3402 can self-expand after it exits lumen 3404.

In an example, expandable flexible member 3403 can be an expandable flexible net or mesh. In an example, flexible member 3403 can be a porous fabric net or mesh. In an example, flexible member 3403 can be a porous bag. In an example, expandable flexible member 3403 can be a balloon with holes. In an example, flexible member 3403 can be expanded by being filled with a plurality of embolic members. In an example, embolic members can be delivered into flexible member 3403 through lumen (e.g. catheter) 3404 or lumen (e.g. catheter) 3405. In an alternative example, flexible member 3403 can be non-porous. In an example, flexible member 3403 can be expanded by being filled with liquid or gas. In an example, a liquid or gas can be delivered into flexible member 3403 through lumen 3404 or lumen 3405.

In an example, flexible member 3403 can be compressed as it travels through a longitudinal lumen (e.g. catheter) and then be expanded within aneurysm sac 1 after it is released from the lumen. In an example, flexible member 3403 can be folded as it travels through a lumen and then be unfolded within aneurysm sac 1. In an example, flexible member 3403 can be relatively loose or relaxed (in a lower-energy state) as it travels through a lumen and then be stretched or tense (in a higher-energy state) within aneurysm sac 1. In an example, flexible member 3403 can be elastic or stretchable. In an example, flexible member 3403 can be sufficiently elastic or stretchable that it expands when filled with an accumulation of embolic members, but not so elastic or stretchable that it allows embolic members to escape. In an example, flexible member 3403 can be a balloon with holes, wherein the holes are of sufficient size to let liquid escape, but not so large that they let embolic members escape.

In an example, embolic members for filling flexible member 3403 can be a plurality of soft, compressible members such as microsponges or blobs of gel. In an example, embolic members can be a plurality of hard, uncompressible members such as hard polymer spheres or beads. In an example, embolic members can be conveyed into flexible member 3403 through lumen (e.g. catheter) 3404 or lumen (e.g. catheter) 3405. In various examples, embolic members can be conveyed via a liquid flow, a moving belt, a wire loop, or an Archimedes screw.

In an example, this invention can comprise a method in which resilient structure 3402 is expanded first and flexible member 3403 is expanded second. In an example, this invention can comprise a method in which flexible member 3403 is expanded first and resilient structure 3402 is expanded second. In an example, this invention can comprise a method in which flexible member 3403 and resilient structure 3402 are expanded at substantially the same time.

As shown in FIGS. 34 through 36, an aneurysm sac can be irregular in shape. An aneurysm sac with an irregular shape will not be completely filled or spanned by a spherical or ellipsoid mass without stretching the aneurysm walls. In an example, the total volume of an aneurysm sac can be X cubic units (e.g. cubic millimeters). In an example, the maximum volume of the aneurysm which can be filled or spanned by a spherical or ellipsoid mass without stretching the aneurysm walls is Y cubic units (e.g. cubic millimeters). In an example, the total device shown in FIGS. 34 through 36 can fill more of the aneurysm than a spherical or ellipsoid mass because flexible member 3403 is sufficiently flexible to fill or span the irregular perimeter of the aneurysm sac. This can have clinical benefits, such as reducing the chances of recanalization within the aneurysm sac. In an example, this device can fill or span more than 50% of the aneurysm volume which remains unfilled by a sphere or ellipsoid. In an example, this device can fill or span Z cubic units (e.g. cubic millimeters) of the volume of the aneurysm, wherein Z>[Y+0.5 (X−Y)].

In an example, the combination of (a) an outer flexible member 3403 that spans substantially the entire perimeter of the aneurysm sac 1 and (b) an inner resilient structure 3402 can create a device that is sufficiently flexible to substantially fill the entire volume of an irregularly-shaped aneurysm sac, but also sufficiently resilient so as to compress against the aneurysm walls and not slip out of the aneurysm sac.

I claim:

1. A device to occlude an aneurysm comprising:
a longitudinal catheter that is configured to be inserted into a blood vessel, wherein the blood vessel is the parent vessel from which an aneurysm has formed;
a longitudinal flexible embolic member that is configured to travel through the longitudinal catheter and be inserted into the aneurysm sac; and
an expandable toroidal member that is configured to travel through the longitudinal catheter, be inserted into the aneurysm sac, and be expanded within the aneurysm sac, wherein the toroidal member is configured to substantially occlude the aneurysm neck after it is expanded, wherein the longitudinal flexible embolic member is inserted into the aneurysm sac through a central opening of the expandable toroidal member, and wherein the expandable toroidal member prevents the longitudinal flexible member from protruding into the parent vessel of the aneurysm.

2. The device in claim 1 wherein the longitudinal flexible embolic member is a coil.

3. The device in claim 1 wherein the expandable toroidal member is expanded in a radial-expansion plane that is substantively parallel with the plane defined by the central circumference of the aneurysm neck.

4. The device in claim 1 wherein a plurality of embolic members are inserted into the aneurysm sac through the central opening.

5. A device to occlude an aneurysm comprising:
a longitudinal catheter that is configured to be inserted into a blood vessel, wherein this blood vessel is the parent vessel from which an aneurysm has formed; and
a series of connected embolic ellipsoids, wherein these embolic ellipsoids are configured to travel in series through the longitudinal catheter and to be inserted into the aneurysm sac; wherein an embolic ellipsoid has a first orientation as it travels through the longitudinal catheter; wherein an embolic ellipsoid has a second orientation after it exits the longitudinal catheter; wherein in the first orientation the longitudinal axis of the ellipsoid is substantively parallel to the longitudinal axis of the longitudinal catheter; wherein in the second orientation the longitudinal axis of the ellipsoid is substantially perpendicular to its prior orientation traveling through the longitudinal catheter.

6. The device in claim 5 wherein the series of connected embolic ellipsoids form a stack of connected ellipsoids which share a common central axis within the aneurysm sac.

7. The device in claim 5 wherein at least one of the connected ellipsoids has a circumference that is larger than the circumference of the aneurysm neck in order to help keep the structure within the aneurysm sac.

8. The device in claim 5 wherein the ellipsoids are compressed after the ellipsoids exit the catheter.

9. The device in claim 8 wherein the ellipsoids are compressed after the ellipsoids exit the catheter by movement of a wire, fiber, or other longitudinal flexible member that is connected to the ellipsoids.

10. A device to occlude an aneurysm comprising:
a longitudinal catheter that is configured to be inserted into a blood vessel, wherein this blood vessel is the parent vessel from which an aneurysm has formed;
an expandable flexible net or mesh, wherein this expandable flexible net or mesh is configured to travel through the longitudinal catheter and to be inserted into the aneurysm sac, and wherein this net or mesh is sufficiently flexible to substantially conform to the walls of an irregularly shaped aneurysm sac after the net or mesh has been expanded;
a plurality of embolic members, wherein these embolic members are configured to travel through the longitudinal catheter and to be inserted into the net or mesh within the aneurysm sac; wherein these embolic members do not escape from the net or mesh; and wherein the net or mesh is expanded by the accumulation of embolic members inside the net or mesh; and
an expandable resilient structure, wherein this expandable resilient structure is configured to travel through the longitudinal catheter and to be inserted into the aneurysm sac; wherein this structure comes into engaging contact with the central circumference of the aneurysm sac when this structure is expanded; and wherein this structure resists compression after it has been expanded.

11. The device in claim 10 wherein the net or mesh is a wire net or mesh.

12. The device in claim 10 wherein the net or mesh can is a polymer net or mesh.

13. The device in claim 10 wherein the expandable resilient structure is a stent.

14. The device in claim 10 wherein the expandable resilient structure is attached to the net or mesh.

15. The device in claim 10 wherein the expandable resilient structure has a bioadhesive coating which adheres to the aneurysm walls.

16. The device in claim 10 wherein the plurality of embolic members are compressed as they travel through the catheter but expand when released from the catheter.

17. The device in claim 10 wherein the device further comprises a closure mechanism which is integrated into the net or mesh to prevent embolic members from escaping from net or mesh through the opening by which they were inserted into net or mesh.

18. The device in claim 17 wherein the closure mechanism is a drawstring, loop, seal, fusible member, adhesive, snap, clip, valve, or cap.

19. The device in claim 10 wherein the expandable resilient structure has a ring or cylindrical shape.

20. The device in claim 10 wherein the expandable resilient structure has an ellipsoidal shape.
